SEQUENCE LISTING (10 sequences)

SEQ ID NO: 1 - protein, 746 aa, Homo sapiens
KNNVPRLKLSYKEMLESNNVITFNGLANSSSYHTFLLDEERSRLYVGAKDHIFSFDLVNIKDFQKIVWPVSYTRRDECKWAGKDILKECANFIKVLKAYNQTHLYACGTGAFHPICTYIEIGHHPEDNIFKLENSHFENGRGKSPYDPKLLTASLLIDGELYSGTAADFMGRDFAIFRTLGHHHPIRTEQHDSRWLNDPKFISAHLISESDNPEDDKVYFFFRENAIDGEHSGKATHARIGQICKNDFGGHRSLVNKWTTFLKARLICSVPGPNGIDTHFDELQDVFLMNFKDPKNPVVYGVFTTSSNIFKGSAVCMYSMSDVRRVFLGPYAHRDGPNYQWVPYQGRVPYPRPGTCPSKTFGGFDSTKDLPDDVITFARSHPAMYNPVFPMNNRPIVIKTDVNYQFTQIVVDRVDAEDGQYDVMFIGTDVGTVLKVVSIPKETWYDLEEVLLEEMTVFREPTAISAMELSTKQQQLYIGSTAGVAQLPLHRCDIYGKACAECCLARDPYCAWDGSACSRYFPTAKRRTRRQDIRNGDPLTHCSDLHHDNHHGHSPEERIIYGVENSSTFLECSPKSQRALVYWQFQRRNEERKEEIRVDDHIIRTDQGLLLRSLQQKDSGNYLCHAVEHGFIQTLLKVTLEVIDTEHLEELLHKDDDGDGSKTKEMSNSMTPSQKVWYRDFMQLINHPNLNTMDEFCEQVWKRDRKQRRQRPGHTPGNSNKWKHLQENKKGRNRRTHEFERAPRSV

SEQ ID NO: 2 - protein, 749 aa, Xenopus
KNNVPRLRLSYKEMVESNNLITFNGWPNSSSYNTFVLDEERGRLYVGAKDHIFSFNLANVKEFQKILWPVTNIRRDECKWAGKDIVKECANFIKVLKAYNHTHLYACGTGAFHPVCTYIDVGHYPEQDNVFKLEDSFFENGRGKSPYDPKLLTASILIDGELYSGTAADFMGRDFAIFRTLGNHHPIRTEQHDSRWLNDPRFIGAYLVPESDNPEDDKVYFFFRENAIDGEHTGKATHARIGQLCKNDFGGHRSLVNKWTTFLKARLICSVPGPNGIDTHFDELQDVVLMNSKDPKNPVVYAVFTTSSNIFKGSAVCMYSLTDIRRVFLGPYAHRDGPNYQWVPFQGRVPYPRPGTCPRQTFGGFDSTKDLPDEVIMFARSHPAMYNPVFPINNRPIIIKTEVDYQFTQIVVDRVEAEDGQYDVMFIGTDMGTILKVVSVPKETWTDLEEVLLEEMTVFREPTAISAMEISTKQQQLYIGSSVGVSQLPLHRCDVYGKACAECCLARDPYCAWDGSSCSRYFPTAKRRTRRQDIRNGDPLTHCSDLQHQDDPHRQSLEEKIIYGVENSSTFLECSPKSQRALVFWQFQKQNEEKKDEIKVDERKIKTEHGLLLRTLKKRDSGIYYCNAVEHGFMQTLLKVTLEIIDTEHLDELLHKEDEGGDSHKHKEPSNSMSPTQKIWYRDFMQLINHPNLNTMDEFCEQVWKRDRKQRRQKNGNVQVSNTKWKHLQENKKGRNRRTHEFERAPRSV

SEQ ID NO: 3 - protein, 60 aa, Homo sapiens
EAAVATSLGRSNTIGSAPPLRHSWQMPNGHYRRRRRGGPGPGMMCGAVNNLLSDTEEDDK

SEQ ID NO: 4 - protein, 55 aa, Xenopus
EVAVATSLGRSNTIGSAPLRHGWQMPNGHYRRRRKGGIPPVIPSRELSDTEEDDR

SEQ ID NO: 5 - protein, 60 aa, Mus musculus
EAAVATSLGRSNTIGSAPPLRHSWQMPNGHYRRRRRGGPGPGMMCGAVSDLLSDTEEDDK

SEQ ID NO: 6 - protein, 41 aa, Hysterocrates gigas
GVDKAGCRYMFGGCSVNDDCCPRLGCHSLFSYCAWDLTFSD

SEQ ID NO: 7 - DNA, 25 bp, Artificial
  Antisense oligonucleotide
ctatgtgatatgattatttatgacc

SEQ ID NO: 8 - DNA, 25 bp, Artificial
  Antisense morpholino oligonucleotide
atgcaatccaggtcagagagcccat

SEQ ID NO: 9 - DNA, 25 bp, Artificial
  Sense sequence oligonucleotide
atgggctctctgacctggattgcat

SEQ ID NO: 10 - DNA, 25 bp, Artificial
  Sense sequence oligonucleotide
ggtcataaataatcatatcacatag

(19) United States
(12) Patent Application Publication
(10) Pub. No.: US 2012/0321594 A1
(43) Pub. Date: Dec. 20, 2012

(54) METHODS OF CONTROLLING AXON OR DENDRITE DEVELOPMENT OF NEURONAL CELLS

(75) Inventors: Kyonsoo HONG, New York, NY (US); Makoto NISHIYAMA, New York, NY (US); Kazunobu TOGASHI, Isezaki-shi (JP); Melanie J. von Schimmelmann, New York, NY (US)

(73) Assignee: NEW YORK UNIVERSITY, New York, NY (US)

(21) Appl. No.: 13/465,795

(22) Filed: May 7, 2012

Related U.S. Application Data
(60) Provisional application No. 61/483,493, filed on May 6, 2011.

(51) Int. Cl. C12N 5/0793; A61K 35/12; C12Q 1/02; G01N 33/53; A61K 39/395; A61K 38/02; A61K 48/00; A61K 31/675; G01N 33/58; A61P 25/28; A61P 25/18; A61P 27/06; A61P 25/16; A61P 25/14; A61P 25/00

(52) U.S. Cl. 424/93.7; 435/375; 435/29; 435/7.1; 424/130.1; 514/17.7; 514/44 R; 514/90; 435/40.5

(57) ABSTRACT

One aspect of the present invention relates to a method of controlling axon or dendrite development in a neuronal cell population. This method involves providing a neuronal cell population and contacting the neuronal cell population with a modulator of R-type Ca²⁺ channel expression or activity in controlling the neuronal cell population to induce either axon or dendrite development. Another aspect of the present invention relates to a method of treating neuronal injury in a subject. This method involves selecting a subject with neuronal injury mediated by R-type Ca²⁺ channel expression or activity and administering to the selected subject an inhibitor of R-type Ca²⁺ channel expression or activity to induce neuronal axon development under conditions effective to treat the neuronal injury in the subject. Yet another aspect of the present invention relates to a method of screening for agents that modulate R-type Ca²⁺ channel expression or activity.

This application claims the benefit of U.S. Provisional Patent Application Ser. No. 61/483,493, filed May 6, 2011, which is hereby incorporated by reference in its entirety.

This invention was made with government support under grant numbers R01 NS042823 and R01 NS064671 awarded by The National Institutes of Health-National Institute of Neurological Disorders and Stroke. The government has certain rights in this invention.

### FIELD OF THE INVENTION

The present invention relates to controlling axon or dendrite development in neural cells and methods of using the same.

### BACKGROUND OF THE INVENTION

One of the greatest obstacles in developing effective treatment for injuries and degenerative diseases of the nervous system is the poor regenerative capacity of neuronal cells. It is axiomatic that injured mature mammalian central nervous system (“CNS”) neurons neither regenerate nor re-establish functional neuronal circuits, resulting in permanent detrimental malfunction of the CNS. One of the most notable examples is the malfunction after spinal cord injury. Two major impediments to the regeneration of post-injury, mature CNS neurons have been recognized: 1) the states of their intracellular signaling pathways differ from those of the permissive embryonic states and 2) inhibitory factors are present in their microenvironments. Current approaches to promote post-injury CNS repair including those in clinical trials address the second major impediment.

To date, the thrust of research in the field of nerve regeneration has been focused on factors such as chondroitin sulfate, proteoglycans (CSPGs), and myelin associated glycoproteins (MAGs). However, the most recent studies conclude that CSPGs and MAGs are not involved in preventing nerve regeneration in a rodent model of spinal cord injury. Semaphorins, particularly semaphorin 3A (“Sema3A”), is considered to be an environmental inhibitory factor that prevents nerve regeneration. However, the molecular mechanisms by which it does so remain elusive.

Developing neurons become polarized to form axons and dendrites (Arimura & Kaibuchi, “Neuronal Polarity: From Extracellular Signals to Intracellular Mechanisms,” Nat. Rev. Neurosci. 8:194-205 (2007); Barnes & Polleux, “Establishment of Axon-Dendrite Polarity in Developing Neurons,” Annu. Rev. Neurosci. 32:347-381 (2009); Jan, Y. & Jan, L., “The Control of Dendrite Development,” Neuron 40:229-242 (2003)). How immature neurites of developing neurons acquire identities as axons/dendrites is unexplained. It was recently revealed that extracellular signaling molecules induce the axon identity (Adler et al., “UNC-6/Netrin Induces Neuronal Asymmetry and Defines the Site of Axon Formation,” Nat. Neurosci. 9:511-518 (2006); Yi et al., “TGF-β Signaling Specifies Axons During Brain Development,” Cell 142:144-157 (2010)), but it remains unknown whether they also induce the dendrite identity, and if so, how.

As noted supra, how these undifferentiated neurites acquire their dendrite identity is unknown. It is likely that specific, spatiotemporally-regulated extracellular signals activate intracellular signaling pathways to specify the dendrite identity. Regardless of the mechanism underlying inhibition of neuronal regeneration following injury and disease, there exists a need to develop methods and compositions to promote neural regeneration.

The present invention is directed to overcoming these and other deficiencies in the art.

### SUMMARY OF THE INVENTION

One aspect of the present invention relates to a method of controlling axon or dendrite development in a neuronal cell population. This method involves providing a neuronal cell population and contacting the neuronal cell population with a modulator of R-type Ca²⁺ channel expression or activity in controlling the neuronal cell population to induce either axon or dendrite development.

Another aspect of the present invention relates to a method of treating neuronal injury in a subject. This method involves selecting a subject with neuronal injury mediated by R-type Ca²⁺ channel expression or activity and administering to the selected subject an inhibitor of R-type Ca²⁺ channel expression or activity to induce neuronal axon development under conditions effective to treat the neuronal injury in the subject.

Yet another aspect of the present invention relates to a method of screening for agents that modulate R-type Ca²⁺ channel expression or activity. This method involves providing a neuronal cell population, providing one or more candidate agents, and contacting the neural cell population with the one or more candidate agents. Following the contacting, either axonal outgrowth or dendritic outgrowth by neuronal cells in the neuronal cell population is detected. The method also involves identifying the one or more candidate agents as agents that modulate R-type Ca²⁺ channel expression or activity. Detecting is based on when increased axonal outgrowth or dendritic outgrowth is detected compared to when the neuronal cell population is not contacted with the one or more candidate agents.

Polarized neurites, axons and dendrites, form the functional circuitry of the nervous system. Secreted guidance cues often convert the polarity of neuron migration and neurite outgrowth by regulating ion channels. Here, it is shown that secreted Sema3A converts the neurite identity of Xenopus spinal commissural interneurons (“xSCINs”) by activating Caᵥ2.3 channels (“Caᵥ2.3”). Sema3A treatment converted the identity of axons of cultured xSCINs to that of dendrites by recruiting functional Caᵥ2.3. Inhibition of Sema3A signaling prevented both the expression of Caᵥ2.3 and acquisition of the dendrite identity, and inhibition of Caᵥ2.3 function resulted in multiple axon-like neurites of xSCINs in the spinal cord. Further, Sema3A-triggered cGMP production and PKG activity induced, respectively, the expression of functional Caᵥ2.3 and the dendrite identity.

These results reveal a novel mechanism by which a guidance cue controls the identity of neurites during nervous system development. These results demonstrate the importance of the spatiotemporal and dynamic expression patterns of both guidance factors and ion channels in establishing neurite identity during nervous system development. Thus, control of Cav2.3-induced acquisition of the dendrite identity and the simultaneous suppression of the axon identity post-CNS injury represents a new way to promote neuron regeneration. This discovery will allow for the control of neuron regeneration both in vivo and in vitro and provides a significant advance in the treatment of subjects with neural injury.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-1B show cross-sectional views of Xenopus spinal cord to illustrate aspects of Xenopus spinal commissural intemeuron (“xSCIN”) polarization. FIG. 1A shows a series of cross-sectional views of the Xenopus laevis spinal cord illustrating the in vivo polarization process of Xenopus spinal commissural interneurons (“xSCINs”). Initially, at approximately embryonic stage 22 (FIG. 1A, far left), a single axon extends toward the ventral midline from the soma positioned dorsolaterally. Then at approximately stage 26 (FIG. 1A, center), the axon passes the midline. Once it passes the midline, it bifurcates and forms longitudinal axon tract at approximately stage 34 (FIG. 1A, far right). At this stage, multiple undifferentiated neurites extend from the initial axon segment toward the pial surface and ventrally into the marginal zone that eventually develop into dendrites (FIG. 1A, far right). FIG. 1B shows cross sectional views of Xenopus spinal cord at stage 32 and 34/36. The left panel of FIG. 1B shows a cross section of a Xenopus spinal cord at stage 32 stained with anti-Sema3A antibodies. As shown, Sema3A is robustly expressed in the ventral marginal zones of the spinal cord, which approximately coincides with the emergence of putative dendrites from the initial axon segment of CINs. The right panel of FIG. 1B shows emergence of putative dendrites from the AIS of spinal cord CINs at stage 34/36.

FIGS. 2A-2E show results demonstrating that Sema3A increases growth cone Caᵥ2.3 currents. FIGS. 2A-2D each show representative traces of Ca²⁺ currents (left) and summaries of the current (I)/voltage (V) relationship (right) in the presence of 500 nM pimozide (PMZ, total HVA currents) (FIG. 2A); 20 μM nimodipine (nimo, N- & R-type (Caᵥ2.3) currents) (FIG. 2B); 20 μM nimo+300 μM Ni²⁺ (N-type currents) (FIG. 2C); and 20 μM nimo+1 μM ω-conotoxin GVIA (CgTX, Caᵥ2.3-type currents) (FIG. 2D). FIG. 2E is a bar graph illustrating average peak Ca²⁺ currents shown in FIGS. 2A-2D. Caᵥ2.3-type currents were enhanced from 56±4 pA to 246±19 pA. Data are mean±s.e.m. (n)=number of growth cones examined for each experimental condition. Significant differences from the control (culture medium) are indicated (*p<0.05; **p<0.01).

FIGS. 3A-3E illustrate results from a computed biophysical growth cone model, which show a 15-mV growth cone membrane depolarization and VDCC kinetics induced by a Sema3A gradient. FIG. 3A shows the configuration for whole-cell growth cone membrane potential measurement. Abbreviations shown are as follows: Gc: growth cone; Rec: recording electrode; Sm: soma; and S: source of gradient (Scale bar, 20 μm). FIG. 3B shows results of a representative growth cone membrane potential measurement in the presence of an 8-fold higher concentration gradient of Sema3A than that which normally induces growth cone hyperpolarization (Nishiyama et al., “Membrane Potential Shifts Caused by Diffusible Guidance Signals Direct Growth-Cone Turning,” Nat. Neurosci. 11:762-771 (2008), which is hereby incorporated by reference in its entirety). FIG. 3C is a graph of results and shows cumulative distribution of growth cone membrane potentials in each condition (+NP-1 (0111): data obtained from growth cones that over-express a mutant Sema3A receptor, NP-1 (0111)). Dotted lines indicate the average membrane potential for Control and Sema3A-treated samples. Significant difference from the control is indicated (**p<0.01). FIG. 3D is a graph of results that shows normalized, growth cone membrane potential-dependent changes of Ca²⁺ inward currents (A/Ca) through each VDCC subtype.

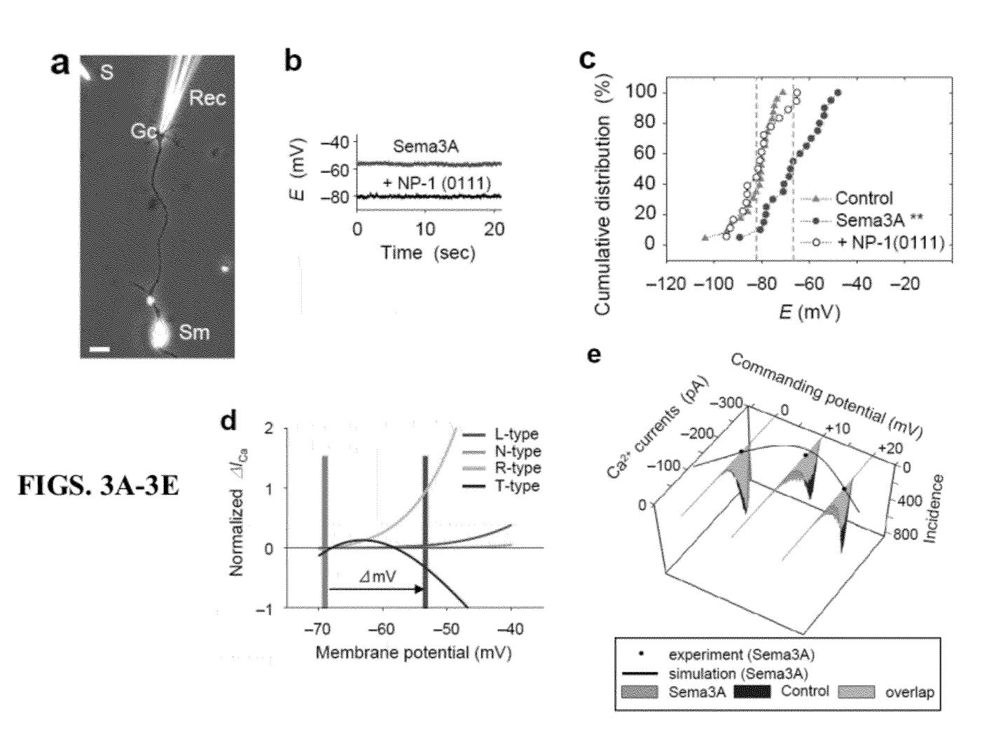

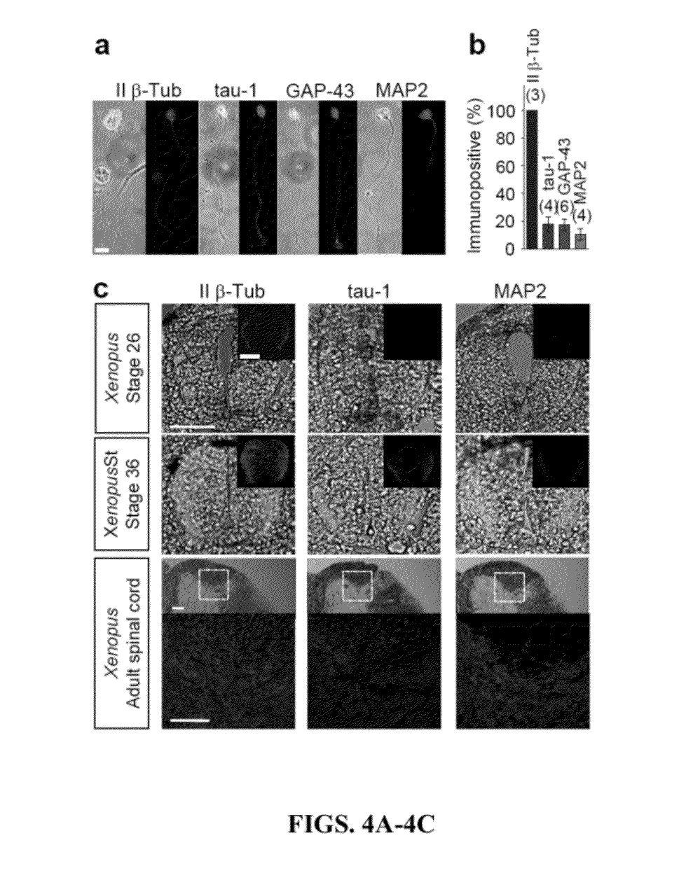

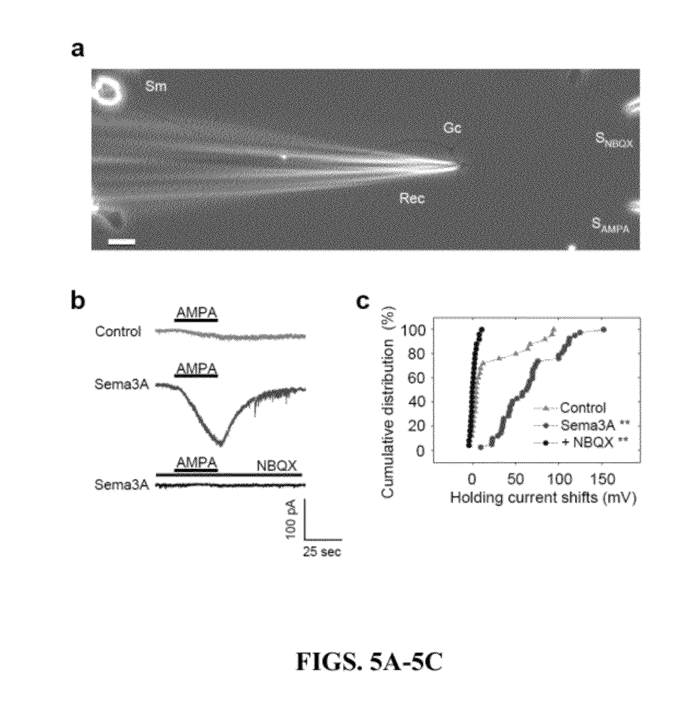

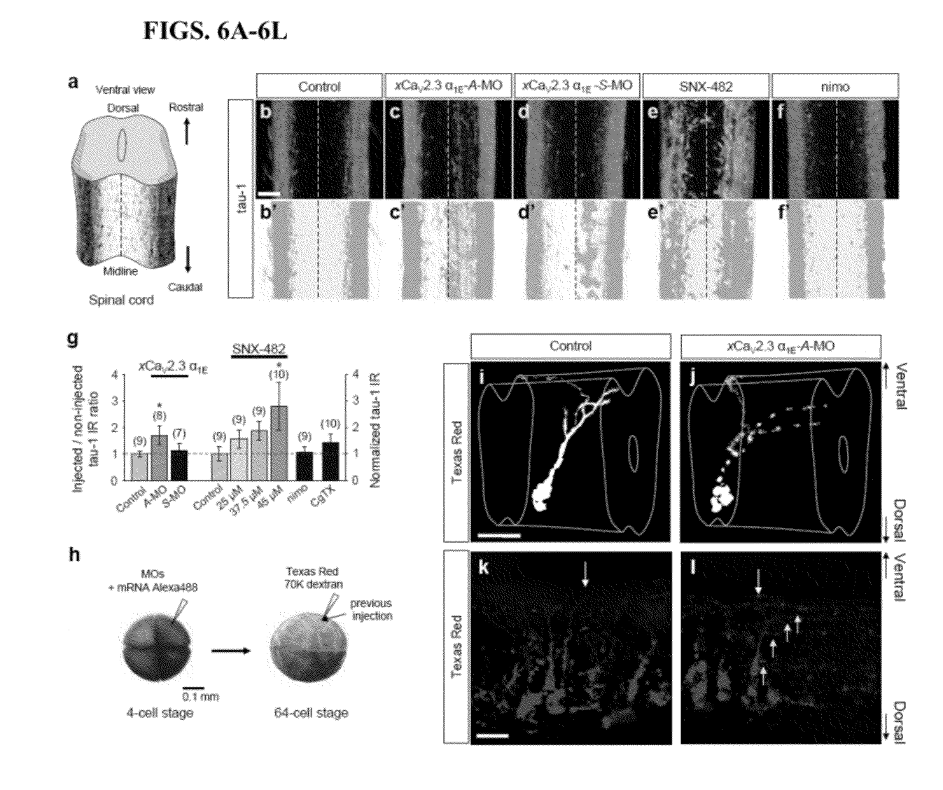

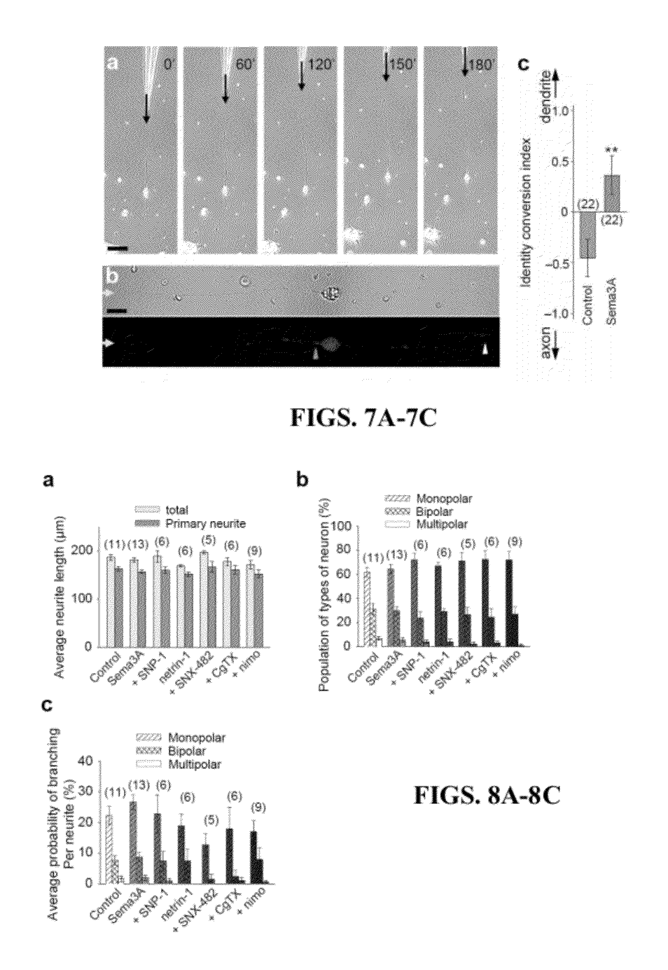

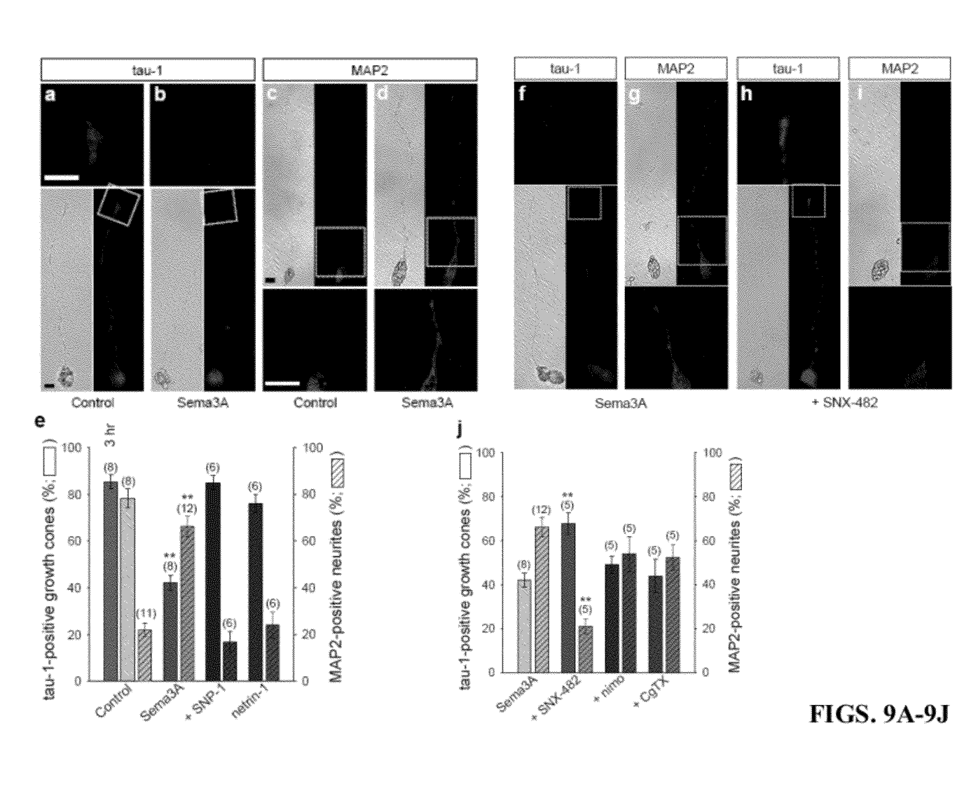

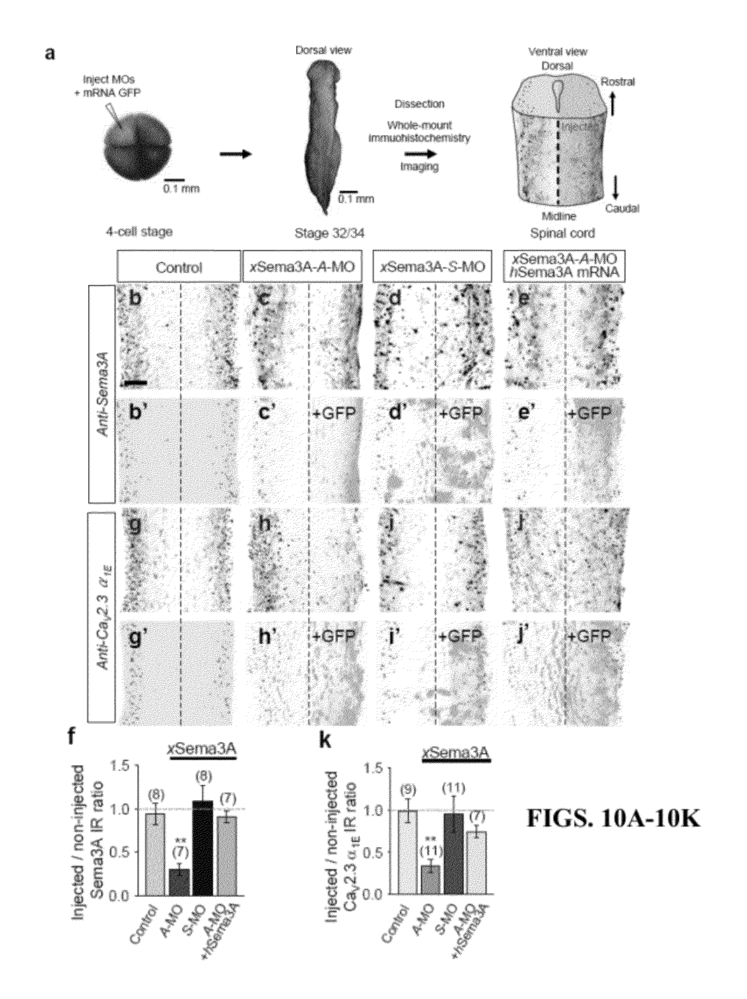

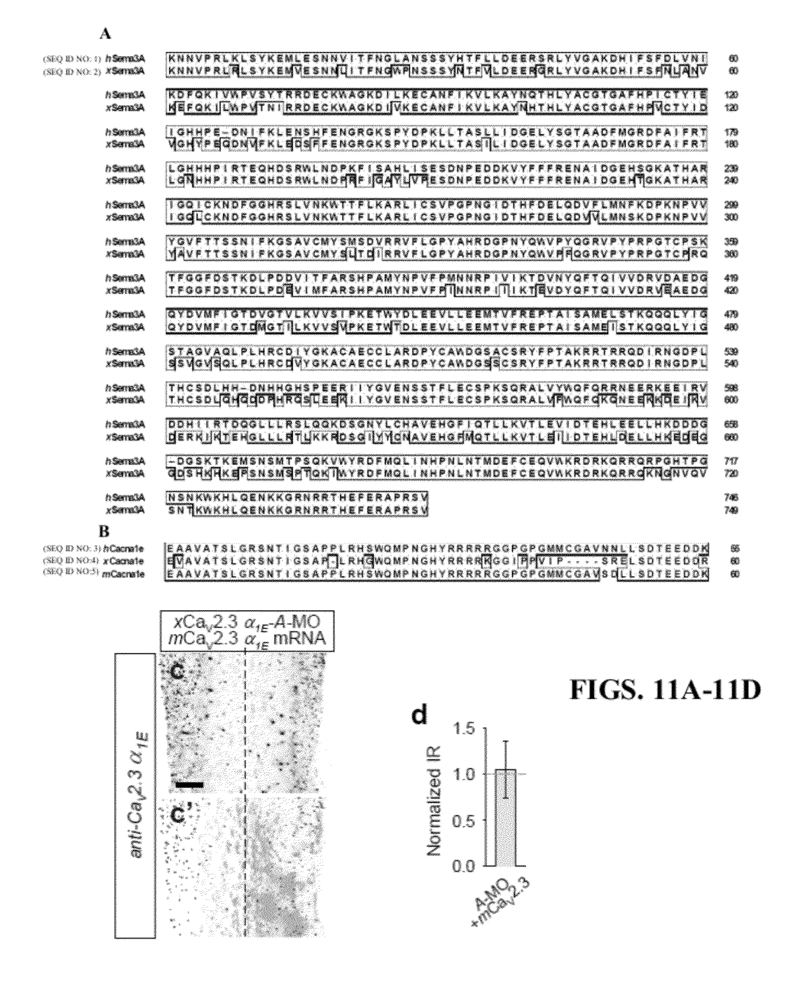

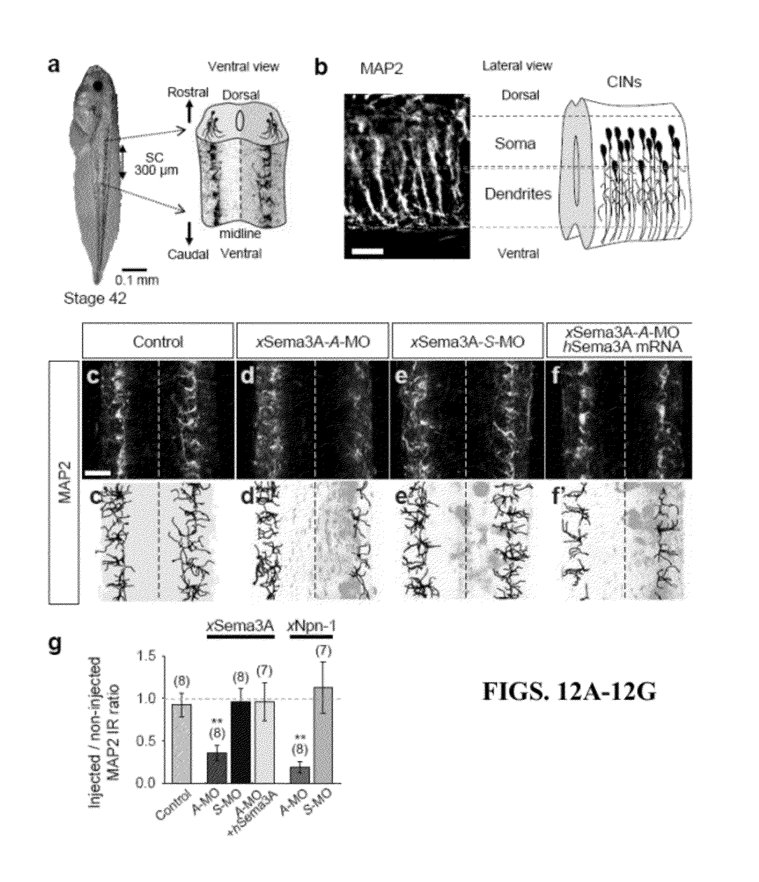

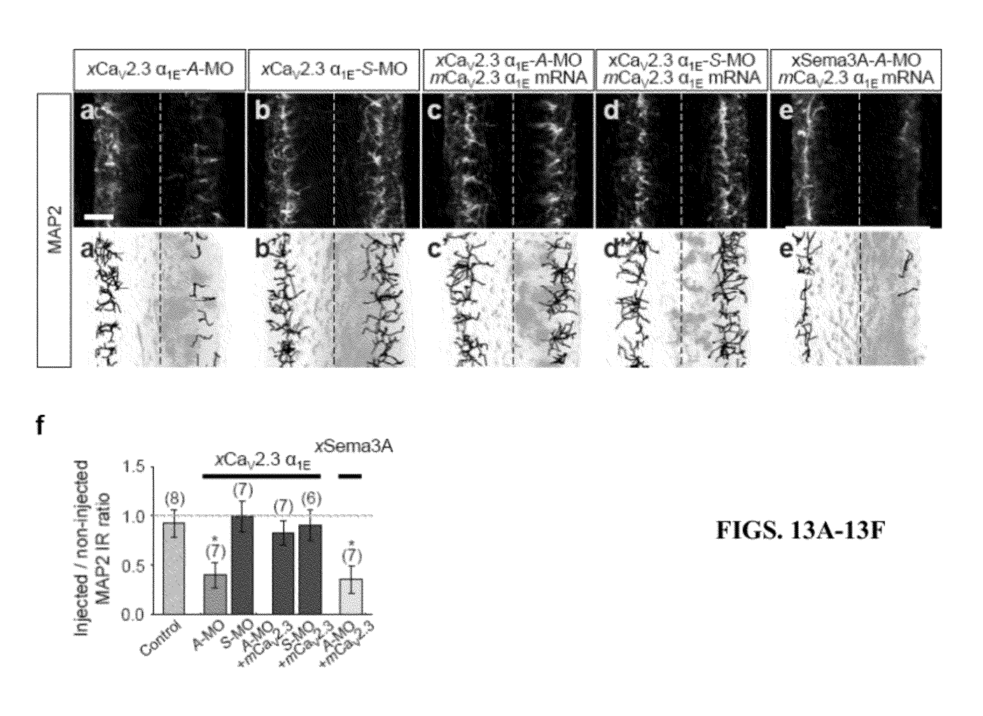

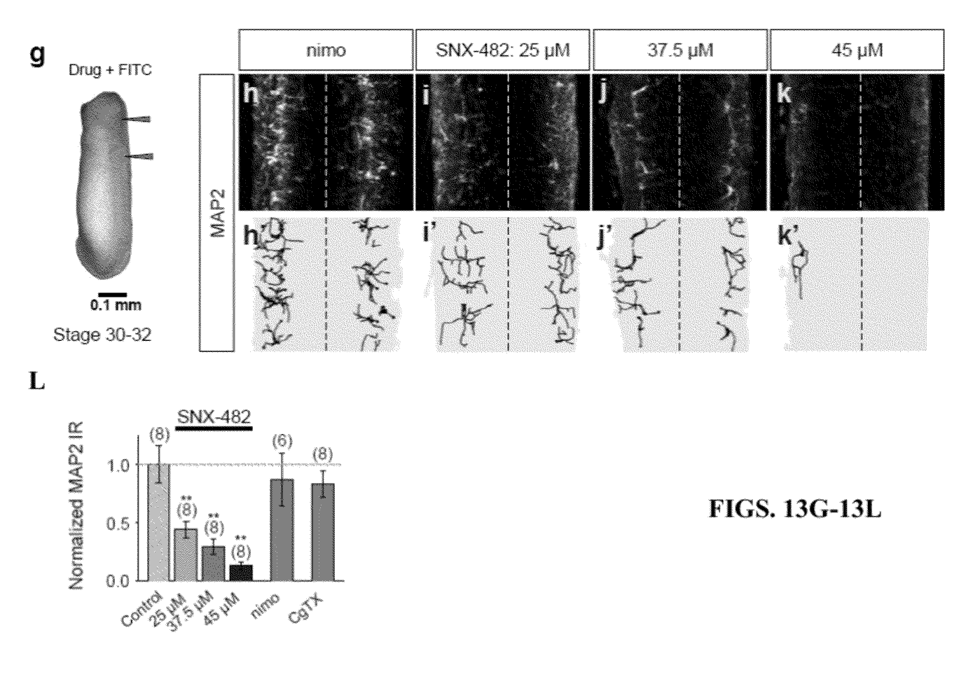

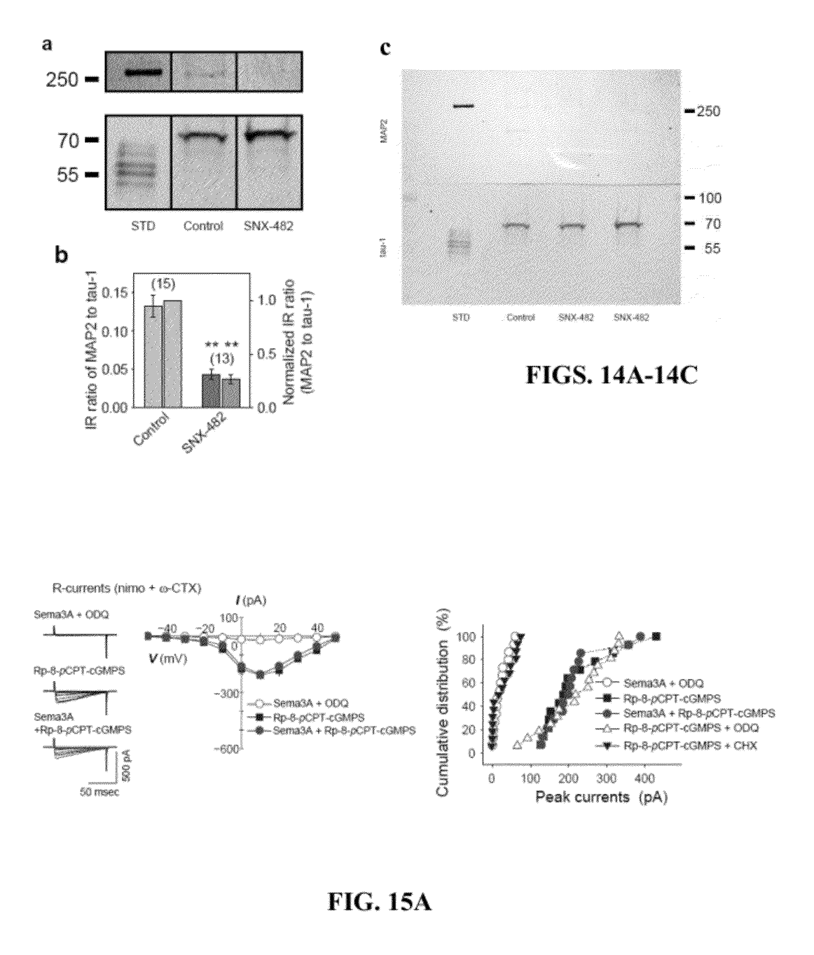

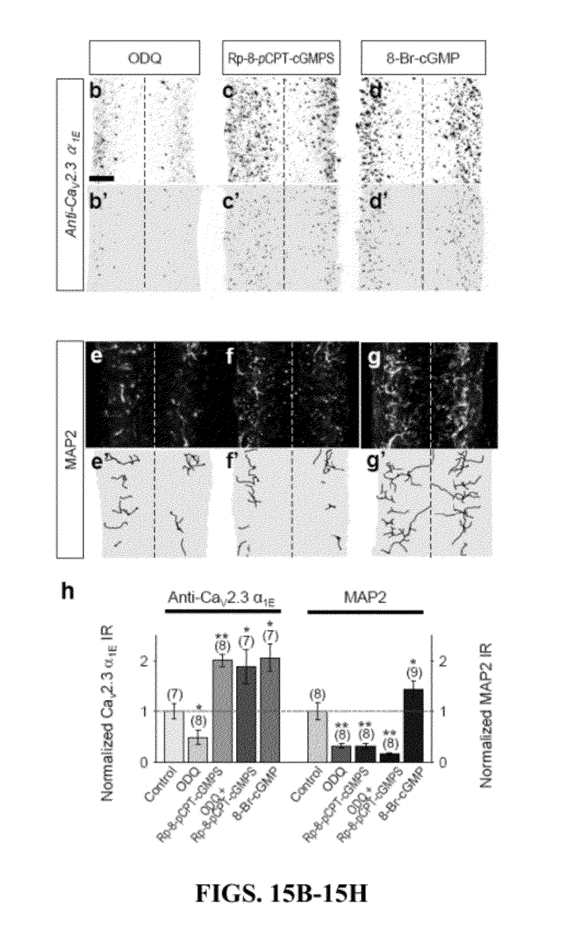

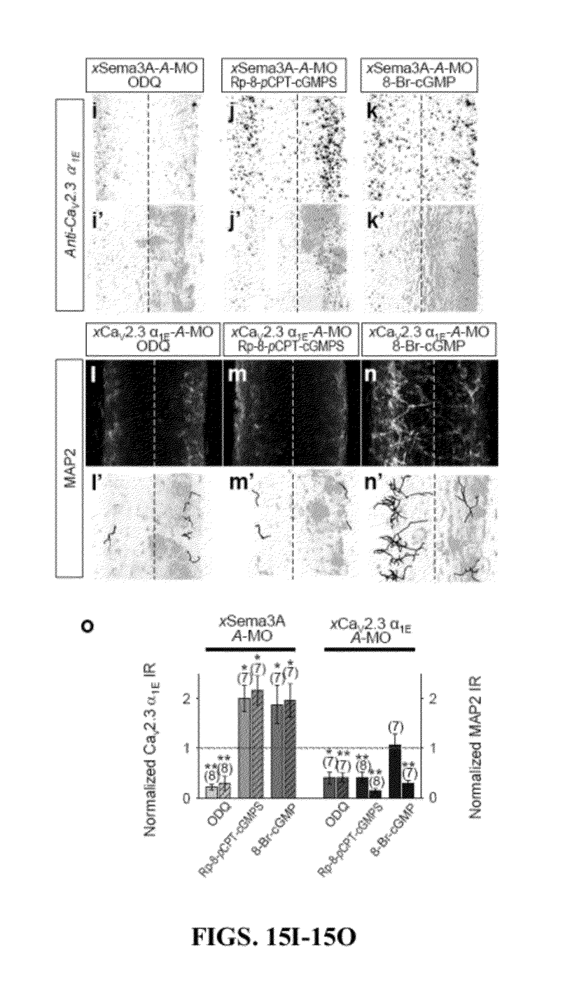

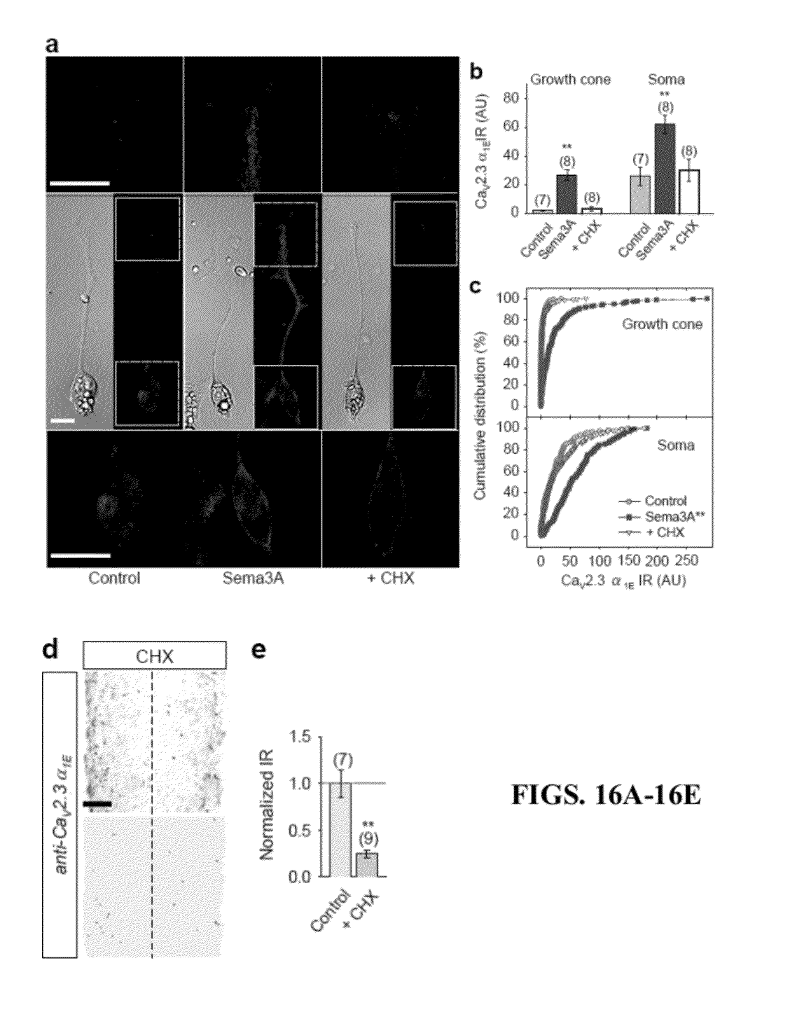

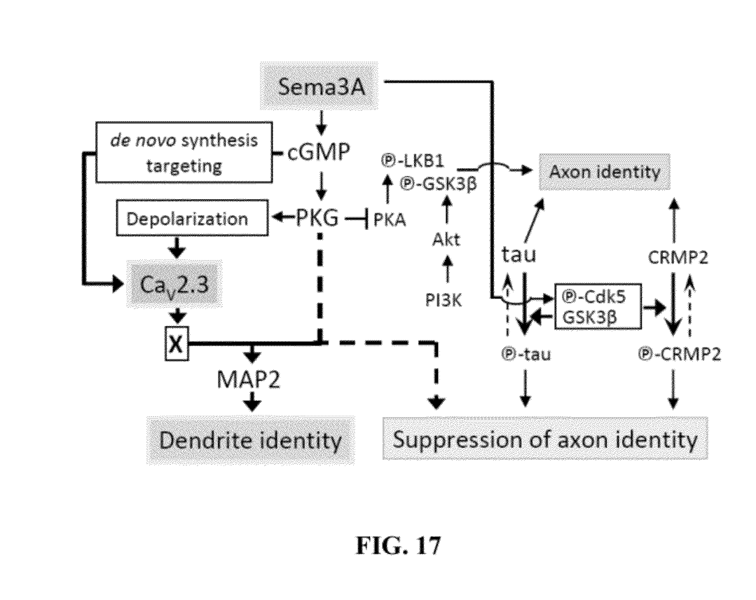

VDCC kinetics and growth cone membrane potentials are readjusted to those that would occur in the culture medium environment (Nishiyama et al., “Membrane Potential Shifts Caused by Diffusible Guidance Signals Direct Growth-Cone Turning,” Nat. Neurosci. 11:762-771 (2008), which is hereby incorporated by reference in its entirety). The model predicts that increased Ca²⁺ currents are contributed by Caᵥ2.3 (R-type) during Sema3A-induced growth cone depolarization (brown), while the contribution from T-type channels decreases due to their voltage-dependent inactivation. FIG. 3E is a graph showing predicted incidence distributions of Caᵥ2.3 currents at each commanding potential in the presence or absence (control) of a Sema3A gradient. The incidence was predicted by 10,000 runs of Monte Carlo simulation based on the voltage-dependent activation and inactivation time constants (Table 1, infra) in the presence or absence of Sema3A. Caᵥ2.3 currents in the control were scaled to those in the presence of Sema3A. The highest incidences at +10 mV were predicted at −265 pA and −254 pA for the control and Sema3A-treated samples, respectively, indicating that a minor portion of the increased Caᵥ2.3 currents (11 pA of 254 pA) in the presence of Sema3A can be attributed to modification of the channel gating properties.

FIGS. 4A-4C show unpolarized neurites of cultured Xenopus spinal neurons and their polarization process in vivo. FIGS. 4A and 4B show images (left: bright field, right: immunofluorescence) (FIG. 4A) and a bar graph of summaries of immunoreactivities (FIG. 4B) of class II β-tubulin (II β-Tub, all neurites), tau-1 and GAP-43 (axons), and MAP2 (dendrites). Xenopus spinal neurons derived from stage 26 embryos were cultured on a poly-1-lysine (5 μg·ml−1) substrate and incubated for 16 hr before they were immunohistochemically stained. Data are mean±s.e.m.; (n)=number of cultures (ca. 300 neurons were examined). FIG. 4C shows images of immunohistochemically-stained cross-sections of a Xenopus spinal cord at different developmental stages showing the sequential appearance of unpolarized neurites (II b-Tub-positive at stage 26), tau-1-positive axons, and MAP2-positive dendrites during development. Fluorescent images of the same sections are shown inset (Scale bars: 20 μm (FIG. 4A) and 50 [m (FIG. 4C)).

FIGS. 5A-5C show results demonstrating that Sema3A induces functional α-amino-3-hydroxy-5-methyl-4-isoxazolepropionic acid receptors (“AMPARs”) in cultured Xenopus CIN growth cones. FIG. 5A shows the configuration used for the measurement of AMPA-induced whole-cell holding current shifts in the growth cone. Abbreviations shown are as follows: Gc: growth cone; Rec: recording electrode; Sm: the soma; SAMPA and SNBQX: sources of gradients of α-amino-3-hydroxy-5-methyl-4-isoxazolepropionic acid (AMPA) and 2,3-dihydroxy-6-nitro-7-sulfamoyl-benzo[f]quinoxaline-2,3-dione (NBQX), respectively (Scale bar, 20 μm). FIG. 5B is a graph showing plots of representative AMPA-induced holding current shifts in control (Control, top) and Sema3A-treated (Sema3A, center) growth cones, and in the presence of NBQX (bottom). FIG. 5C is a graph showing the cumulative distribution of AMPA-induced holding current shifts in growth cones in each condition. Sema3A-treated growth cones (66±5 pA) showed more than three times greater holding current shifts than those in control growth cones (19±7 pA). Significant differences from the control are indicated (**p<0.01).

FIGS. 6A-6L show results demonstrating that Caᵥ2.3 channel activity suppresses the acquisition of the axon identity of xSCINs in vivo. FIG. 6A is a schematic diagram of the spinal cord that was overlaid with an image of the immunofluorescence of tau-1-positive axons. FIGS. 6B-6G show immunofluorescence images (FIGS. 6B-6F) and summaries (FIG. 6G) of tau-1-immunoreactivity (“IR”) of Xenopus spinal cords without injection of Morpholino oligonucleotides (“MOs”) (Control; FIG. 6B, with its bottom panel labeled b′) and with injection of antisense (A-MO; FIG. 6C, with its bottom panel labeled c′) or sense (S-MO; FIG. 6D, with its bottom panel labeled d′) MOs specific to xCaᵥ2.3_{α1E}, or with local administration of SNX-482 (FIG. 6E, with its bottom panel labeled e′) or nimodipine (nimo; FIG. 6F, with its bottom panel labeled f). Signals in orange (shown in each bottom panel of FIGS. 6B-6F, labeled as b′-f′, respectively) indicate the localization of tau-1-IR and are overlaid with the signals from the green fluorescence protein (‘GFP”) marker (green; shown in the bottom panels of FIGS. 6C and 6D, labeled as c′, d′, respectively). The injected side of the spinal cord was normalized against the contralateral, uninjected side. For the uninjected controls, the right sides were normalized against the left sides. For local administration of Ca²⁺ channel antagonists, tau-1-IR of the bilateral sides of the spinal cord was normalized against the average tau-1-IR in control animals. Data are mean±s.e.m.; (n)=number of spinal cords examined. Significant differences from control are indicated (*p<0.05). FIG. 6H is a visualization of individual xSCINs and their neurite projections after the xCaᵥ2.3_{α1E} MO treatment. After the microinjection of MO together with Alex-488 conjugated with 10K-dextran as an injection marker into one dorsal blastomere of four-cell stage embryos, Texas Red conjugated with 70K dextran was injected into one dorsal blastomere of a 64-cell stage. In this preparation, approximately one sixteenth of the spinal neurons in one side of the spinal cord are visualized. FIGS. 6I-6L are images of ventrolateral views of a Xenopus spinal cord showing CIN axons crossing the midline (two white arrows pointing down) in non-treated (FIGS. 6I and 6K) and treated with xCaᵥ2.3-A-MO (FIGS. 6J and 6L). In animals treated with xCaᵥ2.3-AMO, multiple axon-like neurites extending from the initial segment and migrating longitudinally were observed (FIGS. 6J and 6L, yellow arrows pointing up).

FIGS. 7A-7C show results demonstrating that a Sema3A gradient applied to growth cones converts the identity of tau-1-positive axons to MAP2-positive dendrites. FIGS. 7A and 7B are sample images of the experimental configuration (FIG. 7A) and MAP2 staining (FIG. 7B, bright field: top and immunofluorescence: bottom). Arrows on FIG. 7A indicate the direction of the Sema3A gradient. Proximal neurite of a growth cone exposed to the Sema3A gradient (yellow arrows pointing right, FIG. 7B) is robustly MAP2-positive (green arrowhead in center and pointing up, FIG. 7B, bottom), whereas a distal growth cone is stained more at the growth cone (white arrowhead far right and pointing up, putative axon, FIG. 7B). FIG. 7C is a graph showing a summary of the identity conversion induced by the Sema3A gradient. A conversion index (MAP2-negative=−1; MAP2-positive=+1; and not determined=0) is given for each pair of individual neurites. Data are mean±s.e.m.; (n)=number of bipolar neurons examined. Significant difference from the control is indicated (**p<0.01). (Scale bars, 50 μm (FIG. 7A) and 20 μm (FIG. 7B).)

FIGS. 8A-8C are graphs showing results that demonstrate short-term treatment with neither bath-applied guidance molecules nor antagonists significantly affect the morphology of cultured Xenopus spinal CINs. FIG. 8A shows the average total and primary neurite lengths for each treatment—Sema3A: Sema3A (2 U·ml−1); +SNP-1: Sema3A (2 U·ml−1) plus SNP-1 (20 nM); netrin-1: netrin-1 (5 ng·ml−1); +SNX-482: Sema3A (2 U·ml−1) plus SNX-482 (1 μM); +CgTX: Sema3A (2 U·ml−1) plus ω-conotoxin GVIA (1 μM); and +nimo: Sema3A (2 U·ml−1) plus nimodipine (20 μM). FIG. 8B shows percent population of monopolar, bipolar and multipolar neuron types. FIG. 8C shows percent probability that neurites possess branches. Data are mean±s.e.m.; (n)=number of cultures (ca. 150 to 200 neurons) examined.

FIGS. 9A-9J show results demonstrating that Sema3A converts the identity of axons to dendrites by Caᵥ2.3 activity. FIGS. 9A-9D and 9F-9I are bright field (left) and immunofluorescence (right) images of xSCINs cultured on a laminin substrate for six hours under various experimental conditions. Insets show magnified fluorescent images of the same sections in either top or bottom. The images show sections that were untreated (Control; FIGS. 9A and 9C), treated with Sema3A (2 U·ml⁻¹; FIGS. 9B, 9D, 9F, and 9G), or with Sema3A and SNX-482 (1 μM, +SNX482; FIGS. 9H and 9I), and immunostained with tau-1 (FIGS. 9A, 9B, 9F, and 9H) or MAP2 (FIGS. 9C, 9D, 9G, and 9I) (Scale bars, 10 μm). FIGS. 9E and 9J are graphs showing summaries of the percentage of tau-1-positive growth cones and MAP2-positive neurites that emerged from the soma. Neurons were treated with Sema3A and SNP-1 (20 nM, +SNP-1; FIG. 9E), with netrin-1 (5 μg·ml⁻¹; FIG. 9E), with Sema3A and SNX-482 (FIG. 9J), with Sema3A and nimodipine (20 μM, +nimo; FIG. 9J), or with Sema3A and ω-conotoxin GVIA (1 μM, +CgTX; FIG. 9J). The dark grey bar on the far left=tau-1 positive growth cones cultured on a laminin substrate for 3 hrs (FIG. 9E). Data are mean±s.e.m.; (n)=number of cultures (ca. 210 to 450 neurons with neurites of ca. 100⁻250 μm long) examined. Significant differences from Control (FIG. 9E) and Sema3A (FIG. 9J) are indicated (**p<0.01), respectively.

FIGS. 10A-10K show results demonstrating that Sema3A is required for Caᵥ2.3 channel expression in Xenopus spinal cords. FIG. 10A is a schema depicting experimental paradigms. MOs or mRNAs encoding either a GFP marker or human Sema3A (hSema3A) were injected into one dorsal blastomere of a 4-cell stage embryo (left) to treat one side of the spinal cord, while the contralateral side served as an internal control. Whole mount preparations of stage 32 (for Sema3A) or stage 34 (for Caᵥ2.3 channels) tailbud spinal tissues (center) were immunostained with antibodies (anti-Sema3A to detect Xenopus Sema3A (xSema3A) and anti-Caᵥ2.3 to detect Xenopus Caᵥ2.3_{α1E} (xCaᵥ2.3_{α1E}) subunit. Spinal cord schema is overlaid with an immunofluorescence image showing the expression of xSema3A in the non-injected side (left), but not in the injected side (right). FIGS. 10B-10E and 10G-10J are immunofluorescence images of Xenopus spinal cords immunostained for xSema3A (FIGS. 10B-10E) or xCaᵥ2.3_{α1E} (FIGS. 10G-10J), without MO injection (Control; FIGS. 10B and 10G) and with injection of antisense (A-MO; FIGS. 10C, 10E, 10H, and 10J) or sense (S-MO; FIGS. 10D and 10I) MO specific for either xSema3A or xCaᵥ2.3_{α1E}. Signals in red (shown on bottom panels of FIGS. 10B-10E and 10G-10J, labeled b′-e′, g′-j′, respectively) indicate the localization of xSema3A (shown on bottom panels of FIGS. 10B-10E, labeled b′-e′, respectively) and xCaᵥ2.3_{α1E} (shown on bottom panels of FIGS. 10G-10J, labeled g′-j′, respectively) IRs, which are overlaid with signals from the GFP co-injection marker (+GFP, green; shown on bottom panels of FIGS. 10C-10E and 10H-10J, labeled c′-e′ and h′-j′, respectively) (Scale bar, 20 μm). FIGS. 10F and 10K are graphs showing summaries of normalized xSema3A (FIGS. 10B-10E) and xCaᵥ2.3_{α1E} (FIGS. 10G-10J) IRs. Injected sides of spinal cords were normalized to the contralateral, non-injected sides. For non-injected controls, the right sides of spinal cords were normalized against the left sides. Data are mean±s.e.m.; (n)=number of spinal cords examined. Significant differences from control are indicated (**p<0.01).

FIGS. 11A-11D show alignments of epitope site sequences (FIGS. 11A and 11B) and over-expression of mouse Caᵥ2.3_{α1E} (mCaᵥ2.3_{α1E}) in Xenopus spinal cords treated with the A-MO against Xenopus Caᵥ2.3_{α1E} (xCaᵥ2.3_{α1E}-A-MO) (FIGS. 11C and 11D). FIGS. 11A and 11B show amino acid sequences of epitopes recognized by anti-Sema3A (AF1250, SEQ ID NOS:1-2, FIG. 11A) and anti-Caᵥ2.3_{α1E} (H-60, SEQ ID NOs:3-5, FIG. 11B) antibodies. xSema3A and xCaᵥ2.3_{α1E} are 85% and 78% identical, respectively, to their human orthologues. FIG. 11C is an immunofluorescence image of a Xenopus spinal cord stained for xCaᵥ2.3_{α1E} after injection of xCaᵥ2.3_{α1E}-A-MO together with mCaᵥ2.3_{α1E} mRNA. Signals in red (shown in the bottom panel, labeled c′) indicate the localization of Caᵥ2.3_{α1E} IRs, which are overlaid with signals from the GFP injection marker (green) (Scale bar, 20 μm). FIG. 11D is a graph that shows a summary of normalized Caᵥ2.3_{α1E} IRs. Injected sides of spinal cords were normalized against the contralateral, uninjected sides. Data are mean±s.e.m.; n=7 of spinal cords examined.

FIGS. 12A-12G show results demonstrating that Sema3A signaling is required for acquisition of the dendrite identity of putative CINs in vivo. FIG. 12A illustrates a whole-mount preparation of a Xenopus stage 42 tadpole (left) and spinal cord schema overlaid with immunofluorescence images of MAP2-positive dendrites (right). FIG. 12B is an immunofluorescence image of a lateral view of MAP2-positive dendrites (control, left) and schematic diagram of CIN neurite trajectories (right). MAP2-IR reveals dendrites projecting ventrally, indicating that the majority of MAP2-positive dendrites are those of CINs. FIGS. 12C-12F are immunoflluorescence images of spinal cords without MO-injection (Control; FIG. 12C, with its bottom panel labeled c′), or injected with antisense (A-MO; FIG. 12D, with its bottom panel labeled d′) or sense (S-MO; FIG. 12E, with its bottom panel labeled e′) MO specific to xSema3A or xSema3A-A-MO together with hSema3A mRNA (FIG. 12F, with its bottom panel labeled f′). Traces in black (shown in the bottom panels of FIGS. 12C-12F, labeled as c′-f′, respectively) indicate the MAP2-positive dendrite trajectories and are overlaid with signals from the GFP marker (green). FIG. 12G is a graph showing summaries of normalized MAP2-positive volumes of samples treated with either xSema3A- or Xenopus neuropilin-1 (xNpn-1)-MOs. Injected sides of spinal cords were normalized against the contralateral, non-injected sides. For the non-injected controls, the right sides of spinal cords were normalized against the left sides. Data are mean±s.e.m.; (n)=number of spinal cords examined. Significant differences from the control are indicated (**p<0.01) (Scale bars, 20 μm).

FIGS. 13A-13L show results demonstrating Caᵥ2.3 channel function is required for the dendrite identity of xSCINs in vivo. FIGS. 13A-13E and 13H-13K are immuofluorescence images of spinal cords injected with antisense (A-MO; FIG. 13A, with its bottom panel labeled a′) or sense (S-MO; FIG. 13B, with its bottom panel labeled b′) MO specific to xCaᵥ2.3_{α1E}, or xCaᵥ2.3_{α1E}-A-MO or xCaᵥ2.3_{α1E}-S-MO together with mCaᵥ2.3_{α1E} mRNA (FIG. 12C, with its bottom panel labeled c′ and FIG. 13D, with its bottom panel labeled d′) or with xSema3A-A-MO together with mCaᵥ2.3_{α1E} mRNA (FIG. 13E with its bottom panel labeled e′), or with local administration of either nimodipine (nimo; FIG. 13H, with its bottom panel labeled h′) or the indicated concentrations of SNX-482 (FIGS. 13I-13K, with their bottom panels labeled i′, j′, and k′, respectively). Traces in black (shown on the bottom paned of FIGS. 13A-13E and 13H-13K, labeled a′-e′ and h′-k′, respectively) indicate the MAP2-positive neurite trajectories and are overlaid with the signals from the GFP marker (green). FIGS. 13F and 13L are bar graphs showing summaries of normalized MAP2-positive volumes of samples treated with xCaᵥ2.3_{α1E}-MOs, xSema3A-A-MO, or the local administration of Ca²⁺ channel blockers. For animals treated with MOs, injected sides of spinal cords were normalized against the contralateral, non-injected sides. For animals treated with Ca²⁺ channel blockers, the MAP2-positive volumes of the bilateral sides of the spinal cord were normalized against the average MAP2-positive volumes in control animals. FIG. 13G illustrates administration of Ca²⁺ channel blockers (FITC as an injection marker) into the hindbrain and spinal cords of stage 30-32 tailbuds. Data are mean±s.e.m.; (n)=number of spinal cords examined. Significant differences from control are indicated (*p<0.05; **p<0.01) (Scale bars, 20 μm).

FIGS. 14A-14C illustrate results that show increased tau-1 and reduced MAP2 immunoreactivities in SNX- 482-injected spinal cords. FIG. 14A shows representative western blots of homoginates obtained from control spinal cords (Control) or from spinal cords of animals injected locally with SNX-482 probed for MAP2 (top) or tau-1 (bottom) proteins. Standard (STD): microtubule associated protein fraction isolated from bovine brain. FIG. 14B is a bar graph showing the summary of immunoreactivity (IR) ratio (left bars) and normalized IR ratio (right bars) of MAP2 to tau-1 in each experimental condition. MAP2 IRs (AU) were normalized against those of tau-1 in the same specimen. To obtain the normalized IR ratio, each IR ratio was normalized by that of control in the same blot. FIG. 14C is a full scan of the original western blot shown in FIG. 14A. Data are mean±s.e.m. (n)=number of blots (10 to 30 spinal cords for each blot) examined. Significant difference from the control is indicated (**p<0.01).

FIGS. 15A-15O show results demonstrating that Sema3A-induced cGMP causes recruitment and expression of Caᵥ2.3 channels and PKG is required as a co-activator for acquisition of the dendrite identity. FIG. 15A are representative traces of Ca²⁺ currents (left), summary of the current (I)/voltage (V) relationship (center) of Caᵥ2.3 currents and cumulative distribution of peak Caᵥ2.3 currents (right) monitored in cultured xSCIN growth cones. Either ODQ (1 μM), Rp-8-pCPT-cGMPS (2.5 μM) or cycloheximide (CHX, 25 μM) was applied in the bath 30 min before experiments were performed. Sema3A (2,000 U·ml⁻¹ in micropipettes) was applied to the growth cone as a gradient. FIGS. 15B-15G and 15I-15N are immunofluorescence images of Xenopus spinal cords immunostained for xCaᵥ2.3_{α1E} (FIGS. 15B-15D, 15I-15K) or MAP2 (FIGS.15E-15G, 15L-15N), without MO injection (FIGS. 15B-15G) or with injection of A-MO specific for either xSema3A (FIGS. 15I-15K) or xCaᵥ2.3_{α1E} (FIGS. 15L-15N). Signals in red (shown on the bottom panels of FIGS. 15B-15D and 15I-15K, labeled as b′-d′, i′-k′, respectively) and traces in black (shown on the bottom panels of FIGS. 15E-15G and 15L-15N, labeled as e′-g′, l′-n′, respectively) indicate the location of the xCaᵥ2.3_{α1E} and MAP2 IRs, respectively, which are overlaid with signals from the GFP injection marker (green, shown on the bottom panels of FIGS. 15I-15K and 15L-15N, labeled as i′-k′, l′-n′, respectively). ODQ (1 mM), Rp-8-pCPT-cGMPS (0.25 mM) and 8-Br-cGMP (1 mM) were injected into the hindbrain and spinal cord of stage 30-32 tailbud animals (see FIG. 15G). Scale bar, 20 pm. FIGS. 15H and 150 are graphs showing summaries of normalized xCaᵥ2.3_{α1E} IR (FIGS. 15B-15D and 15I-15K) and MAP2-positive volumes (FIGS. 15E-15G and 15L-15N). xCaᵥ2.3_{α1E} IR and MAP2-positive volumes in bilateral sides of the spinal cord treated with ODQ or cGMP analogues were normalized against the average IR and positive volumes in control animals (FIG. 15H). Injected sides of spinal cords were normalized to the average IRs and positive volumes in control animals (FIG. 150). Data are mean±s.e.m. (n)=number of growth cones and spinal cords examined. Significant differences from control are indicated (*p<0.05; **p<0.01).

FIGS. 16A-16E show results demonstrating that Sema3A-induced Caᵥ2.3 channel expression requires protein synthesis. FIG. 16A shows bright field (left) and immunofluorescence (right) images of cultured xSCINs (Insets: magnified fluorescence images either in above or below). Sema3A and +CHX, respectively, indicate treatment with Sema3A (2 U·ml−1) for 10 min or with Sema3A (2 U·ml−1) for 10 min after a one-minute pre-administration of cycloheximide (25 μM). FIG. 16B is a graph showing a summary of Caᵥ2.3_{α1E} immunoreactivity (IR, AU) in each experimental condition. FIG. 16C shows cumulative distribution of Caᵥ2.3_{α1E} IR as in FIG. 16B. FIGS. 16D and 16E are immunofluorescence images (FIG. 16D) and summaries (FIG. 16E) of Caᵥ2.3_{α1E} IR in Xenopus spinal cords either without (Control) or with (+CHX) local injection of cycloheximide (2.5 mM). Caᵥ2.3_{α1E} IR was normalized against that of control animals. For uninjected controls, the right sides of spinal cords were normalized against the left sides. Scale bars, 20 μm; data are mean±s.e.m; (n) =number of cultures (150 to 200 neurons, FIG. 16B) spinal cords examined (FIG. 16E). Significant difference from the control is indicated (**p<0.01).

FIG. 17 is a schematic model depicting Sema3A-induced acquisition of the dendrite identity. Without being bound by theory, Sema3A induces the cGMP-mediated expression of functional Caᵥ2.3 channels and PKGdependent depolarization. Thus, PKG gates Ca²⁺ signaling through Caᵥ2.3 channels to promote the acquisition of the dendrite identity and suppresses the acquisition of the axon identity. Unidentified signaling molecule(s) “X” likely increases MAP2 and decreases unphosphorylated tau proteins.

### DETAILED DESCRIPTION OF THE INVENTION

One aspect of the present invention relates to a method of controlling axon or dendrite development in a neuronal cell population. This method involves providing a neuronal cell population and contacting the neuronal cell population with a modulator of R-type Ca²⁺ channel expression or activity in controlling the neuronal cell population to induce either axon or dendrite development.

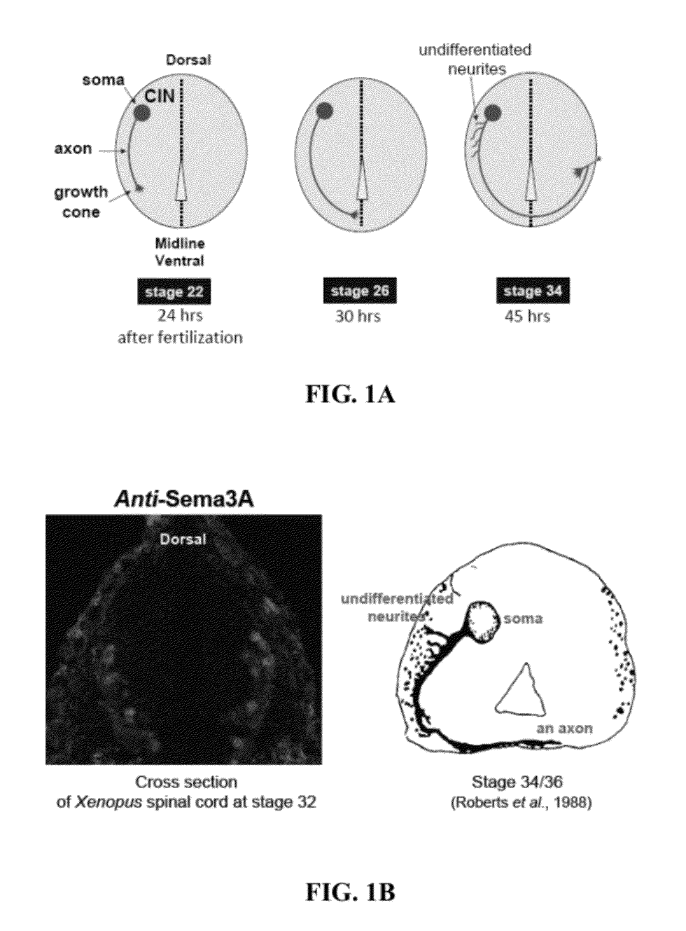

In vivo, acquisition of an identity as either an axon or dendrite occurs at different phases of neuronal polarization. A developing neuron initially acquires an axon extending from the soma and subsequently either a single dendrite, as in C. elegans (Poon et al., “UNC-6/Netrin and its Receptor UNC-5 Locally Exclude Presynaptic Components From Dendrites,” Nature 455:669-673 (2008), which is hereby incorporated by reference in its entirety), or multiple dendrites, as in most vertebrates (Arimura& Kaibuchi, “Neuronal Polarity: From Extracellular Signals to Intracellular Mechanisms,” Nat. Rev. Neurosci. 8:194-205 (2007); Barnes & Polleux, “Establishment of Axon-Dendrite Polarity in Developing Neurons,” Annu. Rev. Neurosci. 32:347-381 (2009), which are hereby incorporated by reference in their entirety). Dendrites develop from undifferentiated neurites that emanate either directly from the soma, e.g., of cerebellar granule cells (Barnes & Polleux, “Establishment of Axon-Dendrite Polarity in Developing Neurons,” Annu. Rev. Neurosci. 32:347-381 (2009), which are hereby incorporated by reference in their entirety), or from the tip of the leading process, e.g., of cortical pyramidal neurons (Barnes & Polleux, “Establishment of Axon-Dendrite Polarity in Developing Neurons,” Annu. Rev. Neurosci. 32:347-381 (2009), which are hereby incorporated by reference in their entirety), or from the axonal initial segment (AIS), e.g., of xSCINs (Roberts et al., “Development and Characterization of Commissural Interneurones in the Spinal Cord of Xenopus laevis Embryos Revealed by Antibodies to Glycine,” Development 103:447-461 (1988); Roberts et al., “The Early Development of Neurons With GABA Immunoreactivity in the CNS of Xenopus laevis Embryos,” J. Comp. Neurol. 261:435-449 (1987), which are hereby incorporated by reference in their entirety). For example, after xSCIN axons cross the midline of the floor plate and bifurcate into ascending and descending axon branches on the opposite side of the lateral fascicle, multiple neurites extend toward the pial surface and ventrally into the marginal zone from the AIS at tailbud stages 32-34 (Roberts et al., “Development and Characterization of Commissural Interneurones in the Spinal Cord of Xenopus laevis Embryos Revealed by Antibodies to Glycine,” Development 103:447-461 (1988), which is hereby incorporated by reference in its entirety). FIG. 1A illustrates the in vivo polarization process of Xenopus spinal commissural interneurons.

Extracellular guidance factors can induce either attraction or repulsion of growth cones (Henley & Poo, “Guiding Neuronal Growth Cones Using Ca²⁺ Signals,” Trends Cell Biol. 14:320-30 (2004); Hong & Nishiyama, “From Guidance Signals to Movement: Signaling Molecules Governing Growth Cone Turning,” Neuroscientist 16:65-78 (2010), which are hereby incorporated by reference in their entirety), or reverse the direction of neuron migration (Guan et al., “Long-Range Ca²⁺ Signaling From Growth Cone to Soma Mediates Reversal of Neuronal Migration Induced by Slit-2,” Cell 129:385-395 (2007), which is hereby incorporated by reference in its entirety) by causing changes in intracellular Ca²⁺ levels, suggesting that external factors trigger early signaling events that initiate cellular processes by regulating ion channels. Sema3A, a secreted molecule, functions in several neuronal processes, including the orientation and maturation of dendrites (Polleux et al., “Semaphorin 3A is a Chemoattractant for Cortical Apical Dendrites,” Nature 404:567-573 (2000); Fenstermaker et al., “Regulation of Dendritic Length and Branching by Semaphorin 3A,” J. Neurobiol. 58:403-412 (2004); Morita et al., “Regulation of Dendritic Branching and Spine Maturation by Semaphorin3a-Fyn Signaling,” J. Neurosci. 26:2971-2980 (2006); Tran et al., “Secreted Semaphorins Control Spine Distribution and Morphogenesis in the Postnatal CNS,” Nature 462:1065-1069 (2009), which are hereby incorporated by reference in their entirety). Sema3A binds to its receptor, neuropilin-1 (Npn-1), in Xenopus spinal neuron growth cones and triggers local production of cGMP by soluble guanylate cyclase (sGC) (Nishiyama et al., “Membrane Potential Shifts Caused by Diffusible Guidance Signals Direct Growth-Cone Turning,” Nat. Neurosci. 11:762-771 (2008), which is hereby incorporated by reference in its entirety). Depending on their intracellular cGMP level ([cGMP]ᵢ), growth cones are either repelled from or attracted toward Sema3A. Low [cGMP]ᵢ induces repulsion (Nishiyama et al., “Membrane Potential Shifts Caused by Diffusible Guidance Signals Direct Growth-Cone Turning,” Nat. Neurosci. 11:762-771 (2008), which is hereby incorporated by reference in its entirety) through the activation of cyclic nucleotide-gated cation channels (Togashi et al., “Cyclic GMP-Gated CNG Channels Function in Sema3A-Induced Growth Cone Repulsion,” Neuron 58:694-707 (2008), which is hereby incorporated by reference in its entirety) and high [cGMP]ᵢ induces attraction (Nishiyama et al., “Membrane Potential Shifts Caused by Diffusible Guidance Signals Direct Growth-Cone Turning,” Nat. Neurosci. 11:762-771 (2008); Song et al., “Conversion of Neuronal Growth Cone Responses from Repulsion to Attraction by Cyclic Nucleotides,” Science 281:1515-1518 (1998), which are hereby incorporated by reference in their entirety) through the activity of an unidentified channel(s).

High level Sema3A expressed at the pial surface of the neocortex contributes to the attraction of dendrites (Polleux et al., “Semaphorin 3A is a Chemoattractant for Cortical Apical Dendrites,” Nature 404:567-573 (2000), which is hereby incorporated by reference in its entirety). Interestingly, a relatively high level of Sema3A is expressed in the marginal zone of the Xenopus spinal cord at tailbud stages 30-32 (Togashi et al., “Cyclic GMP-Gated CNG Channels Function in Sema3A-Induced Growth Cone Repulsion,” Neuron 58:694-707 (2008), which is hereby incorporated by reference in its entirety), immediately before the emergence of putative dendrites from the AIS of spinal cord CINs (Roberts et al., “Development and Characterization of Commissural Interneurones in the Spinal Cord of Xenopus laevis Embryos Revealed by Antibodies to Glycine,” Development 103:447-461 (1988), which is hereby incorporated by reference in its entirety) (FIG. 1B).

In the Examples herein, it is surprisingly shown that, both in vitro and in vivo, Sema3A induces the dendrite identity and simultaneously suppresses the axon identity of xSCINs by regulating cGMP signaling and the expression of functional Caᵥ2.3 channels. It has been accepted that Sema3A induces repulsive signaling in regenerating growth cones of injured neurons. Sema3A expression is enhanced in situ at sites of injury and as a consequence prevents nerve regeneration. However, as described in the Examples herein, it was discovered here that Sema3A induces bifunctional signaling in growth cones in accordance with its concentration and an approximately eight fold higher concentration than that which induces classical repulsive signaling induces attractive signaling. It is also shown that Sema3A recruits different classes of Ca²⁻-conducting ion channels: cyclic nucleotide gated channels (CNGCs) and R-type Ca²⁺ channels (Caᵥ2.3) that, respectively, mediate repulsive and attractive signaling in growth cones. Importantly, blocking one of these Ca²⁺-conducting ion channels converts the polarity of growth cone responses. In the absence of CNCG function, a repulsive Sema3A concentration induces growth cone attraction, whereas an attractive concentration induces repulsion in the absence of Caᵥ2.3 function. Cyclic GMP mediates growth cone repulsion and PKG mediates attraction. Further, a high, but not a low, concentration of Sema3A causes the conversion of pre-formed axons to dendrites by the induction of functional Caᵥ2.3. Sema3A achieves this through a tandem signaling pathway. Cyclic GMP triggered by Sema3A induces de novo synthesis of Caᵥ2.3 and their targeting to growth cone plasma membrane. Coincident PKG-mediated membrane depolarization and Ca²⁺ signaling through Caᵥ2.3 cause the conversion of axons to dendrites. Notably, this is the first demonstration that the Sema3A guidance molecule determines the polarity of neurite specification, much less through this pathway.

In one embodiment according to the present invention, the R-type Ca²⁺ channel is a Caᵥ2.3 channel.

In one embodiment according to the present invention, the neuronal cell population is contacted with a modulator of R-type Ca²⁺ channel expression or activity where the modulator is an inhibitor of R-type Ca²⁺ channel expression or activity, thereby inducing axon development in the neuronal cell population.

Inhibitors according to the present invention include compounds which are R-type Ca²⁺ channel antagonists or blockers. Such inhibitors inhibit R-type Ca²⁺ channel activity. As used herein, an inhibitor of R-type Ca²⁺ channel activity is an agent which interferes with or prevents the activity of R-type Ca²⁺ channel. An activity inhibitor may interfere with the ability of the R-type Ca²⁺ channel to function. An activity inhibitor may be an agent which competes with a naturally occurring activator of R-type Ca²⁺ channel function. Alternatively, the activity inhibitor may bind to the R-type Ca²⁺ channel at a site that may, for example, cause a conformational change in the R-type Ca²⁻ channel, thereby precluding binding of or interaction with the natural activator. Alternatively, an activity inhibitor may interfere with a component upstream or downstream of the R-type Ca²⁺ channel, but which interferes with the activity of the R-type Ca²⁺ channel.

Inhibitors of R-type Ca²⁺ channel expression or activity according to the present invention include inhibitors which modulate the expression or activity of the R-type Ca²⁺ channel either by directly interacting with R-type Ca²⁺ channel (or nucleic acid molecules encoding the R-type Ca²⁺ channel, for example) (i.e., direct inhibition) or by acting on components of the biological pathway in which the R-type Ca²⁺ channel participates (i.e., indirect inhibition). An example of an indirect inhibitor of R-type Ca²⁺ channel expression or activity is a Sema3A inhibitor, which would not act directly on the R-type Ca²⁺ channel expression or activity, but is part of a biological pathway in which the R-type Ca²⁺ channel participates, as described herein.

In one embodiment, the modulator is an inhibitor of R-type Ca²⁺ channel activity that directly interacts with the R-type Ca²⁺ channel. In another embodiment, the modulator is an inhibitor that inhibits R-type Ca²⁺ channel expression by directly interacting with a nucleic acid molecule encoding the R-type Ca²⁺ channel.

In one embodiment, the inhibitor is selected from the group consisting of a nucleic acid molecule, an inhibitory peptide, an antibody, and a small molecule.

Suitable inhibitors of R-type Ca²⁺ channel expression or activity according to the present invention include SNX-482 and/or Nickel ions (Ni²⁺). In one embodiment, the inhibitor is SNX-482. SNX-482 is a peptide inhibitor of the activity of R-type voltage-gated calcium channels. SNX-482 can be isolated from the venom of the African tarantula, Hysterocrates gigas. The peptide acts at presynaptic sites and selectively inhibits the activity of R-type Ca²⁺ channel with high affinity and in a voltage-dependent manner. Structurally, SNX-482 has a similar size and cysteine disulfide bond arrangement with two other spider toxins, hanatoxin (which can inhibit voltage-gated potassium channels) and gramotoxin SIA (an inhibitor of voltage gated calcium channels isolated from the venom of Chilean tarantula Grammostola spatulata or Phrixotrichus spatulata), that selectively block calcium channels by altering their gating. Conserved residues necessary for inhibitory function include cysteine residues necessary for forming disulfide bridges that confer the tertiary structure of the peptide. An example of the sequence for SNX-482 is Gly-Val- Asp-Lys-Ala-Gly-Cys-Arg-Tyr-Met-Phe-Gly-Gly-Cys-Ser-Val-Asn-Asp-Asp-Cys-Cys- Pro-Arg-Leu-Gly-Cys-His-Ser-Leu-Phe-Ser-Tyr-Cys-Ala-Trp-Asp-Leu-Thr-Phe-Ser-Asp (SEQ ID NO: 6).

One of skill in the art will recognize that conservative amino acid substitutions may be made without affecting structure and function of SNX-482. A conservative amino acid substitution refers to an amino acid substitution in which the substituted amino acid residue is of similar charge as the replaced residue and is of similar or smaller size than the replaced residue. Conservative substitutions of amino acids include substitutions made amongst amino acids within the following groups: (a) the small non-polar amino acids, A, M, I, L, and V; (b) the small polar amino acids, G, S, T and C; (c) the amido amino acids, Q and N; (d) the aromatic amino acids, F, Y and W; (e) the basic amino acids, K, R and H; and (f) the acidic amino acids, E and D. Substitutions which are charge neutral and which replace a residue with a smaller residue may also be considered “conservative substitutions” even if the residues are in different groups (e.g., replacement of phenylalanine with the smaller isoleucine). Conservative amino acid substitution also includes the use of amino acid analogs or variants.

As noted above, in accordance with the present invention, inhibitors of R-type Ca²⁺ channel expression or activity include inhibitors selected from the group consisting of a nucleic acid molecule, an inhibitory polypeptide, an antibody, and a small molecule, each of which is described in more detail below.

Exemplary nucleic acid R-type Ca²⁺ channel inhibitors include antisense RNAs or RNAi, such as short interfering RNAs (siRNA), short hairpin RNAs (shRNA), and microRNAs.

The use of antisense methods to inhibit the in vivo translation of genes and subsequent protein expression is well known in the art (e.g., U.S. Pat. No. 7,425,544 to Dobie et al.; U.S. Pat. No. 7,307,069 to Karras et al.; U.S. Pat. No. 7,288,530 to Bennett et al.; U.S. Pat. No. 7,179,796 to Cowsert et al., which are hereby incorporated by reference in their entirety). Antisense nucleic acids are nucleic acid molecules (e.g., molecules containing DNA nucleotides, RNA nucleotides, or modifications (e.g., modifications that increase the stability of the molecule, such as 2′-O-alkyl (e.g., methyl) substituted nucleotides) or combinations thereof) that are complementary to, or that hybridize to, at least a portion of a specific nucleic acid molecule, such as an mRNA molecule (see e.g., Weintraub, H. M., “Antisense DNA and RNA,” Scientific Am. 262:40-46 (1990), which is hereby incorporated by reference in its entirety). The antisense nucleic acid molecule hybridizes to its corresponding target nucleic acid molecule, such as the R-type Ca²⁺ channel mRNA, to form a double-stranded molecule, which interferes with translation of the mRNA, as the cell will not translate a double-stranded mRNA. Antisense nucleic acids used in the methods of the present invention are typically at least 10-12 nucleotides in length, or at least 15, 20, 25, 50, 75, or 100 nucleotides in length. The antisense nucleic acid can also be as long as the target nucleic acid with which it is intended to form an inhibitory duplex. Antisense nucleic acids can be introduced into cells as antisense oligonucleotides, or can be produced in a cell in which a nucleic acid encoding the antisense nucleic acid has been introduced, for example, using gene therapy methods.

In one embodiment, the antisense oligonucleotide is designed to target a nucleotide sequence beginning at the start codon of the R-type Ca²⁺ channel mRNA (e.g., Caᵥ2.3_{α1E} mRNA). In one embodiment the antisense oligonucleotide comprises the nucleic acid sequence 5′-CTA TGT GAT ATG ATT ATT TAT GAC C-3′ (SEQ ID NO: 7). In one embodiment, the antisense oligonucleotide is an antisense morpholino oligonucleotide (A-MO). As will be understood, suitable antisense oligonucleotides according to the present invention can be developed by using complementary oligonucleotides to the sense strand of the target mRNA (e.g., human Caᵥ2.3, GenBank Accession No. NP_{—}001192222; NP_{—}001192223; NP_{—}000712 and Xenopus Caᵥ2.3, GenBank Accession No. ACR81595, which are hereby incorporated by reference in their entirety).

siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of the R-type Ca²⁺ channel nucleotide sequence (e.g., human Caᵥ2.3, GenBank Accession No. NP_{—}001192222; NP_{—}001192223; NP_{—}000712, and Xenopus Caᵥ2.3, GenBank Accession No. ACR81595, which are hereby incorporated by reference in their entirety). siRNA molecules are typically designed to target a region of the mRNA target approximately 50-100 nucleotides downstream from the start codon. Upon introduction into a cell, the siRNA complex triggers the endogenous RNA interference (RNAi) pathway, resulting in the cleavage and degradation of the target mRNA molecule. Various improvements of siRNA compositions, such as the incorporation of modified nucleosides or motifs into one or both strands of the siRNA molecule to enhance stability, specificity, and efficacy, have been described and are suitable for use in accordance with this aspect of the invention (see e.g., WO2004/015107 to Giese et al.; WO2003/070918 to McSwiggen et al.; WO1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety).

Short or small hairpin RNA molecules are similar to siRNA molecules in function, but comprise longer RNA sequences that make a tight hairpin turn. shRNA is cleaved by cellular machinery into siRNA and gene expression is silenced via the cellular RNA interference pathway.

Nucleic acid aptamers that specifically bind to R-type Ca²⁺ channels are also useful in the methods of the present invention. Nucleic acid aptamers are single-stranded, partially single-stranded, partially double-stranded, or double-stranded nucleotide sequences, advantageously a replicatable nucleotide sequence, capable of specifically recognizing a selected non-oligonucleotide molecule or group of molecules by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides, and nucleotides comprising backbone modifications, branchpoints, and non-nucleotide residues, groups, or bridges. Nucleic acid aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof and/or corresponding complementary sequence, promoter, or primer-annealing sequence needed to amplify, transcribe, or replicate all or part of the aptamer molecule or sequence.

R-type Ca²⁺ channel inhibitors of the present invention also include inhibitory peptides. Suitable inhibitory peptides of the present invention include short peptides based on the sequence of the R-type Ca²⁺ channel that exhibit inhibition of R-type Ca²⁺ channel binding and direct biological antagonist activity. The amino acid sequence of a human R-type Ca²⁺ channel from which inhibitory peptides may be derived is the amino acid sequence of Caᵥ2.3 (GenBank Accession No. NP_{—}001192222; NP_{—}001192223; NP_{—}000712, which is hereby incorporated by reference in their entirety). The amino acid sequence of a Xenopus R-type Ca²⁺ channel from which inhibitory peptides may be derived is the amino acid sequence of Caᵥ2.3 (GenBank Accession No. ACR81595, which is hereby incorporated by reference in their entirety).

Suitable inhibitory peptides of the present invention include those that bind, preferably, specifically to the R-type Ca²⁺ channel. Such inhibitory peptides may be chemically synthesized using known peptide synthesis methodology or may be prepared and purified using recombinant technology. Such peptides are usually at least about 4 amino acids in length, but can be anywhere from 4 to 100 amino acids in length. Such peptides may be identified without undue experimentation using well known techniques. Techniques for screening peptide libraries for peptides that are capable of specifically binding to a polypeptide target, in this case the R-type Ca²⁺ channel, are well known in the art (see e.g., U.S. Pat. No. 5,556,762 to Pinilla et al.; U.S. Pat. No. 5,750,373 to Garrard et al.; U.S. Pat. No. 4,708,871 to Geysen; U.S. Pat. No. 4,833,092 to Geysen; U.S. Pat. No. 5,223,409 to Ladner et al.; U.S. Pat. No. 5,403,484 to Ladner et al.; U.S. Pat. No. 5,571,689 to Heuckeroth et al.; U.S. Pat. No. 5,663,143 to Ley et al.; and PCT Publication Nos. WO84/03506 to Geysen and WO84/03564 to Geysen, which are hereby incorporated by reference in their entirety).

In one embodiment of the present invention, the R-type Ca²⁺ channel inhibitor is an antibody. An antibody of the present invention encompasses any immunoglobulin molecule that specifically binds to an epitope of the R-type Ca²⁺ channel. As used herein, “epitope” refers to a region of the R-type Ca²⁺ channel protein that is recognized by the isolated antibody and involved in mediating the binding activity or function of the R-type Ca²⁺ channel. Suitable R-type Ca²⁺ channel antibodies and methods of making the same are known and are commercially available (e.g., H-60, sc-28618, Santa Cruz Biotechnology, Inc.).

The epitope recognized by the antibody of the present invention may be a linear epitope, i.e., the primary structure of the amino acid sequence of the R-type Ca²⁺ channel. Alternatively, the epitope recognized by the isolated antibody of the present invention is a non-linear or conformational epitope, i.e., the tertiary or quaternary structure of the R-type Ca²⁺ channel protein.

As used herein, the term “antibody” is meant to include intact immunoglobulins derived from natural sources or from recombinant sources, as well as immunoreactive portions (i.e., antigen binding portions) of intact immunoglobulins. The antibodies of the present invention may exist in a variety of forms including, for example, polyclonal antibodies, monoclonal antibodies, intracellular antibodies, antibody fragments (e.g., Fv, Fab and F(ab)2), as well as single chain antibodies (scFv), chimeric antibodies and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-5883 (1988); Bird et al, “Single-Chain Antigen-Binding Proteins,” Science 242:423-426 (1988), which are hereby incorporated by reference in their entirety).

In one embodiment, the R-type Ca²⁺ channel inhibitor is a human monoclonal antibody or an active binding fragment thereof Methods for monoclonal antibody production may be carried out using techniques well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Generally, the process involves obtaining immune cells (lymphocytes) from the spleen of a mammal which has been previously immunized with the antigen of interest (i.e., an epitope of the R-type Ca²⁺ channel) either in vivo or in vitro.

The antibody-secreting lymphocytes are then fused with myeloma cells or transformed cells, which are capable of replicating indefinitely in cell culture, thereby producing an immortal, immunoglobulin-secreting cell line. Fusion with mammalian myeloma cells or other fusion partners capable of replicating indefinitely in cell culture is achieved by standard and well-known techniques, for example, by using polyethylene glycol (PEG) or other fusing agents (Milstein and Kohler, “Derivation of Specific Antibody-Producing Tissue Culture and Tumor Lines by Cell Fusion,” Eur. J. Immunol. 6:511 (1976), which is hereby incorporated by reference in its entirety). The immortal cell line, which is preferably murine, but may also be derived from cells of other mammalian species, is selected to be deficient in enzymes necessary for the utilization of certain nutrients, to be capable of rapid growth, and have good fusion capability. The resulting fused cells, or hybridomas, are cultured, and the resulting colonies screened for the production of the desired monoclonal antibodies. Colonies producing such antibodies are cloned, and grown either in vivo or in vitro to produce large quantities of antibody.

Alternatively, monoclonal antibodies can be made using recombinant DNA methods, as described in, e.g., U.S. Pat. No. 4,816,567 to Cabilly et al., which is hereby incorporated by reference in its entirety. The polynucleotides encoding a monoclonal antibody are isolated from mature B-cells or hybridoma cells, for example, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, generate monoclonal antibodies. Alternatively, recombinant monoclonal antibodies or fragments thereof of the desired species can be isolated from phage display libraries (McCafferty et al., “Phage Antibodies: Filamentous Phage Displaying Antibody Variable Domains,” Nature 348:552-554 (1990); Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety).

The polynucleotide(s) encoding a monoclonal antibody can further be modified using recombinant DNA technology to generate alternative antibodies. For example, the constant domains of the light and heavy chains of a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

The monoclonal antibody of the present invention can be a humanized antibody. Humanized antibodies contain minimal sequences from non-human (e.g., murine) antibodies within the variable regions. Such antibodies are used therapeutically to reduce antigenicity and human anti-mouse antibody responses when administered to a human subject. In practice, humanized antibodies are typically human antibodies with minimum to no non-human sequences.

An antibody can be humanized by substituting the complementarity determining region (CDR) of a human antibody with that of a non-human antibody (e.g., mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988), which are hereby incorporated by reference in their entirety). The humanized antibody can be further modified by the substitution of additional residues either in the Fv framework region and/or within the replaced non-human residues to refine and optimize antibody specificity, affinity, and/or capability.

Human antibodies can be produced using various techniques known in the art. For example, immortalized human B lymphocytes immunized in vitro or isolated from an immunized individual that produce an antibody directed against a target antigen can be generated (see e.g., Reisfeld et al., MONOCLONAL ANTIBODIES AND CANCER THERAPY 77 (Alan R. Liss ed., 1985) and U.S. Pat. No. 5,750,373 to Garrard, which are hereby incorporated by reference in their entirety). Also, human antibodies can be selected from a phage library that expresses human antibodies (Vaughan et al., “Human Antibodies with Sub-Nanomolar Affinities Isolated from a Large Non-immunized Phage Display Library,” Nature Biotechnology 14:309-314 (1996); Sheets et al., “Efficient Construction of a Large Nonimmune Phage Antibody Library: The Production of High-Affinity Human Single-Chain Antibodies to Protein Antigens,” Proc. Natl. Acad. Sci. U.S.A. 95:6157-6162 (1998); Hoogenboom et al., “By-passing Immunisation. Human Antibodies From Synthetic Repertoires of Germline VH Gene Segments Rearranged In Vitro,” J. Mol. Biol. 227:381-8 (1992); Marks et al., “By-passing Immunization. Human Antibodies from V-gene Libraries Displayed on Phage,” J. Mol. Biol. 222:581-97 (1991), which are hereby incorporated by reference in their entirety). Human antibodies can also be made in transgenic mice containing human immunoglobulin loci that are capable upon immunization of producing the full repertoire of human antibodies in the absence of endogenous immunoglobulin production. This approach is described in U.S. Pat. No. 5,545,807 to Surani et al.; U.S. Pat. No. 5,545,806 to Lonberg et al.; U.S. Pat. No. 5,569,825 to Lonberg et al.; U.S. Pat. No. 5,625,126 to Lonberg et al.; U.S. Pat. No. 5,633,425 to Lonberg et al.; and U.S. Pat. No. 5,661,016 to Lonberg et al., which are hereby incorporated by reference in their entirety

Procedures for raising polyclonal antibodies are also well known. Typically, such antibodies can be raised by administering the peptide or polypeptide containing the epitope of interest subcutaneously to New Zealand white rabbits which have been bled to obtain pre-immune serum. The antigens can be injected in combination with an adjuvant. The rabbits are bled approximately every two weeks after the first injection and periodically boosted with the same antigen three times every six weeks. Polyclonal antibodies are recovered from the serum by affinity chromatography using the corresponding antigen to capture the antibody. This and other procedures for raising polyclonal antibodies are disclosed in Ed Harlow and David Lane, USING ANTIBODIES: A LABORATORY MANUAL (Cold

Spring Harbor Laboratory Press, 1988), which is hereby incorporated by reference in its entirety.

In addition to whole antibodies, the present invention encompasses binding portions of such antibodies. Such binding portions include the monovalent Fab fragments, Fv fragments (e.g., single-chain antibody, scFv), single variable V_{H} and V_{L} domains, and the bivalent F(ab′)₂ fragments, Bis-scFv, diabodies, triabodies, minibodies, etc. These antibody fragments can be made by conventional procedures, such as proteolytic fragmentation procedures, as described in James Goding, MONOCLONAL ANTIBODIES: PRINCIPLES AND PRACTICE 98-118 (Academic Press, 1983) and Ed Harlow and David Lane, ANTIBODIES: A LABORATORY MANUAL (Cold Spring Harbor Laboratory, 1988), which are hereby incorporated by reference in their entirety, or other methods known in the art.

The present invention also encompasses the use of bispecific humanized antibodies or bispecific antigen-binding fragments (e.g., F(ab′)₂) which have specificity for R-type Ca²⁺ channel and a molecule expressed on a target cell (e.g., a neuronal cell). Techniques for making bispecific antibodies are common in the art (Brennan et al., “Preparation of Bispecific Antibodies by Chemical Recombination of Monoclonal Immunoglobulin G1 Fragments,” Science 229:81-3 (1985); Suresh et al, “Bispecific Monoclonal Antibodies From Hybrid Hybridomas,” Methods in Enzymol. 121:210-28 (1986); Traunecker et al., “Bispecific Single Chain Molecules (Janusins) Target Cytotoxic Lymphocytes on HIV Infected Cells,” EMBO J. 10:3655-3659 (1991); Shalaby et al., “Development of Humanized Bispecific Antibodies Reactive with Cytotoxic Lymphocytes and Tumor Cells Overexpressing the HER2 Protooncogene,” J. Exp. Med. 175:217-225 (1992); Kostelny et al, “Formation of a Bispecific Antibody by the Use of Leucine Zippers,” J. Immunol. 148: 1547-1553 (1992); Gruber et al., “Efficient Tumor Cell Lysis Mediated by a Bispecific Single Chain Antibody Expressed in Escherichia coli,” J. Immunol. 152:5368-74 (1994); and U.S. Pat. No. 5,731,168 to Carter et al., which are hereby incorporated by reference in their entirety). Generally, bispecific antibodies are secreted by triomas (i.e., lymphoma cells fuse to a hybridoma) and hybrid hybridomas. The supernatants of triomas and hybrid hybridomas can be assayed for bispecific antibody production using a suitable assay (e.g., ELISA), and bispecific antibodies can be purified using conventional methods. These antibodies can then be humanized according to methods known in the art. Humanized bispecific antibodies or a bivalent antigen-binding fragment of the bispecific antibody having binding specificity for R-type Ca²⁺ channel and an antigen expressed on a target cell, provides a cell-specific targeting approach.

It may further be desirable, especially in the case of antibody fragments, to modify the antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

The present invention also encompasses the nucleic acid molecules that encode the R-type Ca²⁺ channel antibodies of the invention. The nucleic acids may be present in whole cells, in a cell lysate, or in a partially purified or substantially pure form (i.e., purified away from other cellular components or other contaminants).

Nucleic acids encoding the antibodies of the present invention can be obtained using standard molecular biology techniques. For antibodies expressed by hybridomas, cDNAs encoding the light and heavy chains of the antibody made by the hybridoma can be obtained by standard PCR amplification or cDNA cloning techniques. For antibodies obtained from an immunoglobulin gene library (e.g., using phage display techniques), the nucleic acid encoding the antibody can be recovered from the library.

Preferred nucleic acid molecules of the invention are those encoding the V_{H} and V_{L} sequences of R-type Ca²⁺ channel monoclonal antibodies. Once DNA or cDNA fragments encoding V_{H} and V_{L} segments are obtained, these DNA fragments can be further manipulated by standard recombinant DNA techniques, for example to convert the variable region genes to full-length antibody chain genes, to Fab fragment genes, or to a scFv gene. In these manipulations, a V_{L}- or V_{H}-encoding DNA fragment is operatively linked to another DNA fragment encoding another protein, such as an antibody constant region or a flexible linker. The isolated DNA encoding the V_{H} region can be converted to a full-length heavy chain gene by operatively linking the V_{H}-encoding DNA to another DNA molecule encoding heavy chain constant regions (CH1, CH2, and CH3). The sequences of human heavy chain constant region genes are known in the art (see e.g., Kabat et al., Sequences of Proteins of Immunological Interest, 5^{th} ed., U.S. Department of Health and Human Services, NIH Publication No. 91-3242 (1991), which is hereby incorporated by reference in its entirety) and DNA fragments encompassing these regions can be obtained by standard PCR amplification. The heavy chain constant region can be an IgG1, IgG2, IgG3, IgG4, IgA, IgE, IgM, or IgD constant region, but most preferably is an IgG1 or IgG4 constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as a Fab light chain gene) by operatively linking the V_{L}-encoding DNA to another DNA molecule encoding the light chain constant region. The sequences of human light chain constant region genes are known in the art (see e.g., Kabat et al., Sequences of Proteins of Immunological Interest, 5^{th} ed., U.S. Department of Health and Human Services, NIH Publication No. 91-3242 (1991), which is hereby incorporated by reference in its entirety) and DNA fragments encompassing these regions can be obtained by standard PCR amplification. The light chain constant region can be a kappa or lambda constant region, but most preferably is a kappa constant region.

To create a scFv gene, the V_{H}- and V_{L}-encoding DNA fragments are operatively linked to another fragment encoding a flexible linker such that the V_{H} and V_{L} sequences can be expressed as a contiguous single-chain protein, with the V_{H} and V_{L} regions joined by the flexible linker (see e.g., Bird et al., “Single Chain Antigen-Binding Proteins,” Science 242:423-426 (1988); Huston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-5883 (1988); McCafferty et al., “Phage Antibodies: Filamentous Phage Displaying Antibody Variable Domains,” Nature 348:552-554 (1990), which are hereby incorporated by reference in their entirety).

Antibody mimics are also suitable inhibitors for use in accordance with the present invention. A number of antibody mimics are known in the art including, without limitation, those known as monobodies, which are derived from the tenth human fibronectin type III domain (¹⁰Fn3) (Koide et al., “The Fibronectin Type III Domain as a Scaffold for Novel Binding Proteins,” J. Mol. Biol. 284:1141-1151 (1998); Koide et al., “Probing Protein Conformational Changes in Living Cells by Using Designer Binding Proteins: Application to the Estrogen Receptor,” Proc. Natl. Acad. Sci. USA 99:1253-1258 (2002), each of which is hereby incorporated by reference in its entirety); and those known as affibodies, which are derived from the stable alpha-helical bacterial receptor domain Z of staphylococcal protein A (Nord et al., “Binding Proteins Selected from Combinatorial Libraries of an alpha-helical Bacterial Receptor Domain,” Nature Biotechnol. 15(8):772-777 (1997), which is hereby incorporated by reference in its entirety).

In one embodiment according to the present invention, the neuronal cell population is contacted with a modulator is an agent that induces R-type Ca²⁺ channel expression or activity, thereby inducing dendrite development in the neuronal cell population. In one embodiment, the agent that induces R-type Ca²⁺ channel expression or activity comprises Sema3A, cGMP, or both Sema3A and cGMP. This agent is sometimes referred to as an agonist of R-type Ca²⁺ channel expression or activity.

Sema3A administration is known for use in other therapeutic applications and suitable doses, modes of administration, and formulations are also known (see e.g., U.S. Pat. No. 8,088,735 to Neufeld et al., which is hereby incorporated by reference in its entirety).

Cyclic GMP refers to cyclic guanosine 3′,5-monophosphate. This compound is a cyclic nucleotide of guanosine monophosphate which functions at the cellular level to mediate the action of certain hormones across the cellular membrane. cGMP is a cyclic nucleotide derived from the guanosine triphosphate (GTP).

In one embodiment, the contacting according to the present invention is carried out in vivo. In another embodiment the contacting according to the present invention is carried out in vitro.

In one embodiment, providing a neuronal cell population according to the present invention includes providing one or more neuronal cells whose cell body was originally located in the central nervous system (e.g., endogenously located in the CNS), but which have been explanted and cultured ex vivo, as well as the progeny of such cells. Examples of such neurons are motor neurons, interneurons and sensory neurons including retinal ganglion cells, dorsal root ganglion cells, and neurons of the spinal cord.

In another embodiment of the according to the present invention, providing a neuronal cell population includes providing a population of pluripotent stem cells and inducing production of neurons from the population of pluripotent stem cells.

Another discovery of the present invention, particularly related to the Sema3A-induced conversion of axons to dendrites, is the demonstration that guidance molecules concurrently guide and specify extending neurites to be either axons or dendrites as required. This concurrent action of guidance molecules is an efficient mechanism for the formation of synapses. Extending neurites migrate long distances without making synapses before they reach their final synaptic target cells. It is, therefore, advantageous for axons to grow rapidly while they are migrating. In contrast, when growth cones make synapses with their cognate target cells, the conversion of rapidly growing axons to slow-growing dendrites would be advantageous for the formation of precise synaptic contacts. The conversion of neurite polarity as needed is important for neurons to perform particular tasks (e.g., growth or synapse formation), particularly for neurons that form en-passant synapses. To date, this idea has not been proposed.

Accordingly, the methods according to the present invention may include contacting the neuronal cell population with a modulator of R-type Ca²⁺ channel expression or activity that induces dendrite development after contacting said cell population with a modulator of R-type Ca²⁺ channel expression or activity that induces axon development. In one embodiment, the method includes treating the neuronal cell population with a modulator of R-type Ca²⁺ channel expression or activity that induces axon development and contacting the treated neuronal cell population with a modulator of R-type Ca²⁺ channel expression or activity that induces dendrite development. Suitable agents that induce dendrite development are described supra.

In one embodiment, the neuronal cell population is contacted with an amount of an inhibitor of R-type Ca²⁺ channel expression or activity suitable to induce axonal outgrowth by one or more neuronal cells in the neuronal cell population. Then, the neuronal cell population is contacted with an amount of an agent that induces R-type Ca²⁺ channel expression or activity suitable to induce dendritic outgrowth by one or more neuronal cells in the neuronal cell population. This includes, for example, contacting the neuronal cell population with a Sema3A inhibitor followed by contacting the neuronal cell population with Sema3A. In one embodiment, the concentration of Sema3A in the neuronal cell population varies by about eight fold between the (i) contacting with an amount of an inhibitor of R-type Ca²⁺ channel expression or activity suitable to induce axonal outgrowth by one or more neuronal cells in the neuronal cell population and (ii) contacting the neuronal cell population with an amount of an agent that induces R-type Ca²⁻ channel expression or activity suitable to induce dendritic outgrowth by one or more neuronal cells in the neuronal cell population.

Another aspect of the present invention relates to a method of treating neuronal injury in a subject. This method involves selecting a subject with neuronal injury mediated by R-type Ca²⁺ channel expression or activity and administering to the selected subject an inhibitor of R-type Ca²⁺ channel expression or activity to induce neuronal axon development under conditions effective to treat the neuronal injury in the subject.

Highly expressed Sema3A at the mature CNS post-injury lesion site attracts regenerating growth cones and concurrently causes the conversion of their axons to slow outgrowth dendrites. These converted, regenerating dendrites are not able to pass the lesion site to re-establish functional neuronal connections.

The data described in the Examples herein indicate that a conventional concept (i.e., removing/neutralizing inhibitory activities to promote nerve regeneration) will not succeed as expected in the field of nerve regeneration. Blocking inhibitory factors does not remarkably ameliorate post-injury effects on the CNS. The present data indicates that spatio-temporally-regulated, well-balanced attractive and repulsive guidance signaling is useful for post-injury regeneration of the CNS. Ion channels that mediate particular guidance signals are identified herein, and specific modulators for these ion channel activities will be therapeutic for various CNS disorders. Thus, the present invention relates to, inter alia, the therapeutic application of R-type Ca²⁺ channel inhibitors or blockers for the treatment of central nervous system injury. Malfunction of Sema3A signaling has been reported to be associated with various neurological disorders such as Alzheimer disease, schizophrenia, and multiple sclerosis. The idea that the identity of neurites can be switched depending on the magnitude of Sema3A signaling can be widely applicable to these neurological disorders as well. Further, because tansplanted pluripotent cells must establish properly functioning neuronal connections despite the presence of adverse environmental factors, another application of the present invention relates to the clinical application of adult stem cells for the treatment of CNS injury and neurodegenerative diseases.

In one embodiment, the neuronal injury is associated with amyotrophic lateral sclerosis (ALS), trigeminal neuralgia, glossopharyngeal neuralgia, Bell's Palsy, myasthenia gravis, muscular dystrophy, progressive muscular atrophy, primary lateral sclerosis (PLS), pseudobulbar palsy, progressive bulbar palsy, spinal muscular atrophy, inherited muscular atrophy, invertebrate disk syndromes, cervical spondylosis, plexus disorders, thoracic outlet destruction syndromes, peripheral neuropathies, prophyria, Alzheimer's disease, Huntington's disease, Parkinson's disease, Parkinson-plus syndrome, multiple system atrophy, progressive supranuclear palsy, corticobasal degeneration, dementia with Lewy bodies, frontotemporal dementia, demyelinating diseases, Guillain-Barré syndrome, multiple sclerosis, Charcot-Marie-Tooth disease, prion disease, Creutzfeldt-Jakob disease, Gerstmann-Sträussler-Scheinker syndrome (GSS), fatal familial insomnia (FFI), bovine spongiform encephalopathy, Pick's disease, epilepsy, AIDS dementia complex, peripheral neuropathy or neuralgia, glaucoma, lattice dystrophy, retinitis pigmentosa, age-related macular degeneration (AMD), photoreceptor degeneration associated with wet or dry AMD, other retinal degeneration, optic nerve drusen, optic neuropathy, optic neuritis, traumatic nerve injury, schizophrenia, Tuberous Sclerosis complex, Autism Spectrum Disorder, or combinations thereof.

The present invention is useful in the treatment of any neural injury (including that caused by neurodegenerative diseases) which will benefit by enhanced axonal growth. Other states which will benefit by the present invention will be apparent to one of ordinary skill in the art.

Suitable inhibitors according to the present invention are described supra.

The methods according to the present invention are useful to treat or reduce the severity of CNS injuries and neurodegenerative disorders described herein. As compared with an equivalent untreated control, symptoms are reduced by (or the degree of prevention is reduced by) at least 5%, 10%, 20%, 40%, 50%, 60%, 70%, 80%, 90%, 95%, or 100%, as measured by any standard technique. Diagnosis of these disorders is well known in the art and involves, for example, the detection of symptoms associated with the disorder (e.g., tremors, impaired cognition, seizures, memory loss, headaches, and agitation), CAT scans, and Magnetic resonance imaging. One skilled in the art will understand that these patients may have been subjected to the same standard tests as described above or may have been identified, without examination, as one at high risk due to the presence of one or more risk factors (e.g., family history or genetic predisposition).

Suitable subjects in accordance with the present invention include mammals. In one embodiment, the subject is a human. In one embodiment, the subject is a rodent.

In accordance with the methods of the present invention, the mode of administering the inhibitor of the present invention, including the use of suitable delivery vehicles, to a subject will vary depending on the type of inhibitor (e.g., nucleic acid molecule, inhibitory peptide, antibody, or small molecule).

In one embodiment, inhibitory nucleic acid molecules (i.e., antisense, siRNA, etc.), nucleic acid molecules encoding an inhibitory peptide, or nucleic acid molecules encoding an antibody or antibody binding fragment according to the present invention may be incorporated into a gene therapy vector to facilitate delivery.

In a preferred embodiment, the gene therapy vector carrying the inhibitory nucleic acid molecule, nucleic acid molecule encoding an inhibitory peptide, or nucleic acid molecule encoding an antibody or antibody binding fragment according to the present invention is an expression vector derived from a virus. Suitable viral vectors include, without limitation, adenovirus, adeno-associated virus, retrovirus, lentivirus, or herpes virus.

Adenoviral vector gene delivery vehicles can be readily prepared and utilized as described in Berkner, “Development of Adenovirus Vectors for the Expression of Heterologous Genes,” Biotechniques 6:616-627 (1988); Rosenfeld et al., “Adenovirus-Mediated Transfer of a Recombinant Alpha 1-Antitrypsin Gene to the Lung Epithelium In Vivo,” Science 252:431-434 (1991); WO 93/07283 to Curiel et al.; WO 93/06223 to Perricaudet et al.; and WO 93/07282 to Curiel et al., which are hereby incorporated by reference in their entirety. Adeno-associated viral gene delivery vehicles can be constructed and used to deliver a gene, including a gene encoding an antibody to cells as described in Shi et al., “Therapeutic Expression of an Anti-Death Receptor-5 Single-Chain Fixed Variable Region Prevents Tumor Growth in Mice,” Cancer Res. 66:11946-53 (2006); Fukuchi et al., “Anti-Aβ Single-Chain Antibody Delivery via Adeno-Associated Virus for Treatment of Alzheimer's Disease,” Neurobiol. Dis. 23:502-511 (2006); Chatterjee et al., “Dual-Target Inhibition of HIV-1 In Vitro by Means of an Adeno-Associated Virus Antisense Vector,” Science 258:1485-1488 (1992); Ponnazhagan et al., “Suppression of Human Alpha-Globin Gene Expression Mediated by the Recombinant Adeno-Associated Virus 2-Based Antisense Vectors,” J. Exp. Med. 179:733-738 (1994); and Zhou et al., “Adeno-Associated Virus 2-Mediated Transduction and Erythroid Cell-Specific Expression of a Human Beta-Globin Gene,” Gene Ther. 3:223-229 (1996), which are hereby incorporated by reference in their entirety. In vivo use of these vehicles is described in Flotte et al., “Stable In Vivo Expression of the Cystic Fibrosis Transmembrane Conductance Regulator With an Adeno-Associated Virus Vector,” Proc. Nat'l. Acad. Sci. 90:10613-10617 (1993) and Kaplitt et al., “Long-Term Gene Expression and Phenotypic Correction Using Adeno-Associated Virus Vectors in the Mammalian Brain,” Nature Genet. 8:148-153 (1994), which are hereby incorporated by reference in their entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, which are hereby incorporated by reference in their entirety.

Retroviral vectors which have been modified to form infective transformation systems can also be used to deliver inhibitory nucleic acid molecules or nucleic acid molecules encoding a desired peptide or antibody to a target cell. One such type of retroviral vector is disclosed in U.S. Pat. No. 5,849,586 to Kriegler et al., which is hereby incorporated by reference in its entirety.

Gene therapy vectors carrying the therapeutic nucleic acid molecule are administered to a subject by, for example, intravenous injection, local administration (U.S. Pat. No. 5,328,470 to Nabel et al., which is hereby incorporated by reference in its entirety) or by stereotactic injection (see e.g., Chen et al. “Gene Therapy for Brain Tumors: Regression of Experimental Gliomas by Adenovirus Mediated Gene Transfer In Vivo,” Proc. Nat'l. Acad. Sci. USA 91:3054-3057 (1994), which is hereby incorporated by reference in its entirety). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. Gene therapy vectors typically utilize constitutive regulatory elements which are responsive to endogenous transcription factors.

Another suitable approach for the delivery of the compounds and/or treatment agents of the present invention, including the inhibitory nucleic acid molecules, the nucleic acid molecules encoding an inhibitory peptide or the inhibitory peptide itself, and the nucleic acid molecules encoding the antibody or the antibodies themselves, involves the use of liposome delivery vehicles.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated inhibitor at the primary target site. This can be accomplished, for example, in a passive manner where the liposome bilayer degrades over time through the action of various agents in the body.

In contrast to passive drug release, active drug release using liposome delivery vehicles can also be achieved. For example, liposome membranes can be constructed to be pH sensitive (see e.g., Wang & Huang, “pH-Sensitive Immunoliposomes Mediate Target-cell-specific Delivery and Controlled Expression of a Foreign Gene in Mouse,” Proc. Nat'l Acad. Sci. USA 84:7851-5 (1987), which is hereby incorporated by reference in its entirety). When liposomes are endocytosed by a target cell, for example, they can be routed to acidic endosomes which will destabilize the liposome and result in drug release. Alternatively, the liposome membrane can be chemically modified such that an enzyme placed as a coating on the membrane slowly destabilizes the liposome.

Different types of liposomes can be prepared using methods known in the art (see e.g., Bangham et al., “Diffusion of Univalent Ions Across the Lamellae of Swollen Phospholipids,” J. Mol. Biol. 13:238-52 (1965); U.S. Pat. No. 5,653,996 to Hsu; U.S. Pat. No. 5,643,599 to Lee et al.; U.S. Pat. No. 5,885,613 to Holland et al.; U.S. Pat. No. 5,631,237 to Dzau & Kaneda; and U.S. Pat. No. 5,059,421 to Loughrey et al., which are hereby incorporated by reference in their entirety).

Yet another approach for delivery of an inhibitory peptide involves the conjugation of the desired peptide or polypeptide to a stabilized polymer to avoid enzymatic degradation of the inhibitory peptide. Conjugated peptides or polypeptides of this type are described in U.S. Pat. No. 5,681,811 to Ekwuribe, which is hereby incorporated by reference in its entirety.

The compounds and/or treatment agents of the present invention can be administered via any standard route of administration known in the art, including, but not limited to, parenteral (e.g., intravenous, intraarterial, intramuscular, subcutaneous injection, intrathecal), oral (e.g., dietary), topical, transmucosal, or by inhalation (e.g., intrabronchial, intranasal or oral inhalation, intranasal drops). Administration may also be carried out using tissue scaffolds (e.g., those described in U.S. Pat. Nos. 7,892,573 and 6,461,629 and the like, which are hereby incorporated by reference in their entirety). Typically, parenteral administration is the preferred mode of administration.

In one embodiment, the administering is carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes.

Compounds and/or treatment agents according to the present invention are formulated in accordance with their mode of administration. For oral administration, for example, the compounds of the present invention are formulated into an inert diluent or an assimilable edible carrier, enclosed in hard or soft shell capsules, compressed into tablets, or incorporated directly into food. Compounds of the present invention may also be administered in a time release manner incorporated within such devices as time-release capsules or nanotubes. Such devices afford flexibility relative to time and dosage. For oral therapeutic administration, the agents of the present invention may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the compound in these compositions may, of course, be varied and may conveniently be between about 2% to about 60% of the weight of the unit. The amount of a compound of the present invention in such therapeutically useful compositions is such that a suitable dosage will be obtained.

Also specifically contemplated are oral dosage forms of the compounds and/or other treatment agents of the present invention. These may be chemically modified so that oral delivery is efficacious. Generally, the chemical modification contemplated is the attachment of at least one moiety to the component molecule itself, where said moiety permits inhibition of proteolysis and uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polypro line (Abuchowski & Davis, “Soluble Polymer-Enzyme Adducts,” in ENZYMES AS DRUGS (Hocenberg & Roberts eds., 1981), which is hereby incorporated by reference in their entirety). Other polymers that could be used are poly-1,3-dioxolane and poly-1,3,6-tioxocane. Preferred for pharmaceutical usage, as indicated above, are polyethylene glycol moieties.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, sucrulose, or saccharin.

When the dosage unit form is a capsule, it may contain, in addition to materials of the above type, a liquid carrier such as a fatty oil.

The compounds of the present invention may also be formulated for parenteral administration. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver compounds and/or treatment agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of compound and/or treatment agents can also be achieved using infusion pump devices such as those described by Medtronic (Northridge, Calif.). Such devices allow continuous infusion of desired compounds avoiding multiple injections and multiple manipulations.

In addition to the formulations described previously, compounds and/or treatment agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Effective doses of the compounds and/or treatment agents of the present invention vary depending upon many different factors, including type and stage of cancer or tumor, mode of administration, target site, physiological state of the patient, other medications or therapies administered, and physical state of the patient relative to other medical complications. Treatment dosages need to be titrated to optimize safety and efficacy. In one embodiment, the R-type Ca²⁺ channel inhibitor is administered at a dose effective to induce or increase axonal outgrowth by a neuronal cell compared to an untreated cell or standard value.

The compounds and/or treatment agents of the present invention can be administered in a single dose or multiple doses. The dosage can be determined by methods known in the art and can be dependent, for example, upon the subject's age, sensitivity, tolerance and overall well-being. Suitable dosages for antibodies can be from about 0.1 mg/kg body weight to about 10.0 mg/kg body weight per treatment.

The compounds and/or treatment agents of the present invention can be administered to an individual (e.g., a human) alone or in conjunction with one or more other treatment agents. Accordingly, the subject treatment may be performed in combination with any other CNS treatment.

In one embodiment, the R-type Ca²⁺ channel inhibitor according to the present invention may also be administered in combination with other therapeutic agents. The R-type Ca²⁺ channel inhibitor may be administered prior to, with, and/or after the other therapeutic agent.

In one embodiment, the R-type Ca²⁺ channel inhibitor is administered in conjunction with a CNS therapeutic. Suitable CNS therapeutics include, but are not limited to, those suitable for the treatment of amyotrophic lateral sclerosis (ALS), trigeminal neuralgia, glossopharyngeal neuralgia, Bell's Palsy, myasthenia gravis, muscular dystrophy, progressive muscular atrophy, primary lateral sclerosis (PLS), pseudobulbar palsy, progressive bulbar palsy, spinal muscular atrophy, inherited muscular atrophy, invertebrate disk syndromes, cervical spondylosis, plexus disorders, thoracic outlet destruction syndromes, peripheral neuropathies, prophyria, Alzheimer's disease, Huntington's disease, Parkinson's disease, Parkinson-plus syndrome, multiple system atrophy, progressive supranuclear palsy, corticobasal degeneration, dementia with Lewy bodies, frontotemporal dementia, demyelinating diseases, Guillain-Barré syndrome, multiple sclerosis, Charcot-Marie-Tooth disease, prion disease, Creutzfeldt-Jakob disease, Gerstmann-Stráussler-Scheinker syndrome (GSS), fatal familial insomnia (FFI), bovine spongiform encephalopathy, Pick's disease, epilepsy, AIDS dementia complex, peripheral neuropathy or neuralgia, glaucoma, lattice dystrophy, retinitis pigmentosa, age-related macular degeneration (AMD), photoreceptor degeneration associated with wet or dry AMD, other retinal degeneration, optic nerve drusen, optic neuropathy, optic neuritis, traumatic nerve injury, schizophrenia, Tuberous Sclerosis complex, Autism Spectrum Disorder, or combinations thereof.

Agents useful in the treatment of the relevant disease or condition will be known to those of skill in the art. For example, in the treatment of ALS, inhibitors can be administered in combination with Riluzole (Rilutek), minocycline, insulin-like growth factor 1 (IGF-1), and/or methylcobalamin. In another example, in the treatment of Parkinson's disease, inhibitors can be administered with L-dopa, dopamine agonists (e.g., bromocriptine, pergolide, pramipexole, ropinirole, cabergo line, apomorphine, and lisuride), dopa decarboxylase inhibitors (e.g., levodopa, benserazide, and carbidopa), and/or MAO-B inhibitors (e.g., selegiline and rasagiline). In a further example, in the treatment of Alzheimer's disease, inhibitors can be administered with acetylcholinesterase inhibitors (e.g., donepezil, galantamine, and rivastigmine) and/or NMDA receptor antagonists (e.g., memantine). As another example, neuronal injury may result from acute spinal cord injury, which may be treated with, inter alia, an anti-inflammatory agent. Exemplary anti-inflammatory agents include nonsteroidal anti-inflammatory drugs (also called NSAIDs) such as COX-2 inhibitors. Other exemplary anti inflammatory agents include corticosteroids. For example, methylprednisolone is a corticosteroid that can be administered for reduction of CNS inflammation. The combination therapies can involve concurrent or sequential administration, by the same or different routes, as determined to be appropriate by those of skill in the art.

In one embodiment, the CNS therapeutic includes a population of stem cells.

In one embodiment, the CNS therapeutic includes an inhibitor of Sema3A. Suitable inhibitors of Sema3A are known (e.g., those described in U.S. Pat. No. 7,244,761 to Kimura et al., which is hereby incorporated by reference in its entirety).

In one embodiment, the administering includes contacting one or more neuronal cells at a neuronal injury site with an inhibitor of R-type Ca²⁺ channel expression or activity, thereby stimulating axon development in the one or more neuronal cells at the neuronal injury site.

Yet another aspect of the present invention relates to a method of screening for agents that modulate R-type Ca²⁺ channel expression or activity. This method involves providing a neuronal cell population, providing one or more candidate agents, and contacting the neural cell population with the one or more candidate agents. Following the contacting, either axonal outgrowth or dendritic outgrowth by neuronal cells in the neuronal cell population is detected. The method also involves identifying the one or more candidate agents as agents that modulate R-type Ca²⁺ channel expression or activity. Detecting is based on when increased axonal outgrowth or dendritic outgrowth is detected compared to when the neuronal cell population is not contacted with the one or more candidate agents.

In one embodiment, an increase in axon outgrowth is detected and the candidate agent is identified as an inhibitor of R-type Ca²⁺ channel expression or activity.

In one embodiment, an increase in dendrite outgrowth is detected and the candidate agent is identified as an agent that induces R-type Ca²⁺ channel expression or activity.

In one embodiment, the detecting includes contacting the neuronal cell population with a reagent suitable to detect one or more markers of axonal and/or dendritic outgrowth. In one embodiment, the reagent is suitable to detect one or more markers of axonal outgrowth, wherein the one or more markers includes tau-1, GAP-43, or both tau-1 and GAP-43. In another embodiment, the reagent is suitable to detect one or more markers of dendritic outgrowth, wherein the one or more markers includes MAP2.

Suitable reagents include antibodies. Useful antibodies to detect one or more markers of axonal and/or dendritic outgrowth are known (e.g., tau-1 (MAB3420, Millipore), GAP43 (Pierce Endogen) and MAP2 (AP18, Thermo Scientific)).

It will be understood that the increased axonal outgrowth or dendritic outgrowth may be detected and compared to when the neuronal cell population is not contacted with the one or more candidate agents by comparison to a control where a neuronal cell population is not contacted with the one or more candidate agents.

In one embodiment, the neuronal cell population is a mammalian neuronal cell population. In another embodiment, the neuronal cell population is a Xenopus neuronal cell population. In one embodiment, the method of screening is carried out in vivo. In another embodiment, the method of screening is carried out in vitro.

### EXAMPLES

The following examples are provided to illustrate embodiments of the present invention but are by no means intended to limit its scope.

### Example 1

### Animals

Frogs (Xenopus laevis) were purchased from NASCO and housed in the animal facility to maintain a colony.

### Example 2

### In vitro Transcription, Embryo Microinjection, and Culture of Primary Xenopus Spinal Neurons

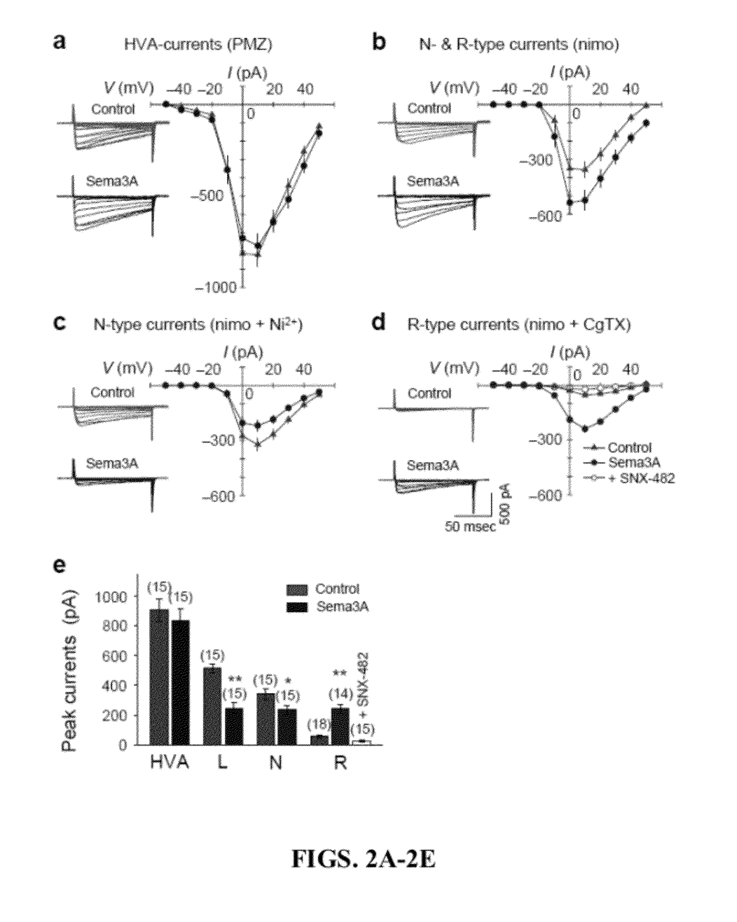

Capped messenger RNAs encoding NP1-0111, hSema3A, mCaᵥ2.3_{α1E}, and green fluorescent protein (GFP) were synthesized with mMESSAGE mMACHINE (Ambion Inc.) and purified on NucAway™ Spin Columns (Ambion Inc.) as described by the manufacturer. Messenger RNAs were injected into one dorsal blastomere of four-cell stage Xenopus embryos (Nishiyama et al., “Membrane Potential Shifts Caused by Diffusible Guidance Signals Direct Growth-Cone Turning,” Nat. Neurosci. 11:762-771 (2008); Togashi et al., “Cyclic GMP-Gated CNG Channels Function in Sema3A-Induced Growth Cone Repulsion,” Neuron 58:694-707 (2008); Nishiyama et al., “Cyclic AMP/GMP-Dependent Modulation of Ca²⁻ Channels Sets the Polarity of Nerve Growth-Cone Turning,” Nature 423:990-995 (2003); Hong et al., “A Ligand-Gated Association Between Cytoplasmic Domains of UNC5 and DCC Family Receptors Converts Netrin-Induced Growth Cone Attraction to Repulsion,” Cell 97:927-941 (1999), which are hereby incorporated by reference in their entirety). Each blastomere was injected with 4 to 8 nl of 0.5 μg·ul⁻¹ NPI-0111, 0.5 μg·μl⁻¹ hSema3A, or 0.5 μg·μl⁻mCaᵥ2.3_{α1E} mRNA and 0.25 μg·μl⁻¹ GFP mRNA as an expression indicator. Primary Xenopus spinal neurons derived from developmental stage 26 embryos were cultured at 23° C.-25° C. for 16-20 hours and used for growth cone electrophysiology (FIGS. 2A-2E; FIGS. 3A-3C) and immunocytochemistry (FIGS. 4A and 4B). To examine the neurite identity and expression of functional AMPA receptor channels (FIGS. 5A-5C), cells were plated on cover glasses that had been coated for four to six hours each with poly-L-lysine (100 μg·ml⁻¹) and laminin (80 μg·ml⁻¹) and incubated for three to four hours at 23° C.-25° C. prior to use.

### Example 3

### Morpholino Oligonucleotides

Antisense morpholino oligonucleotides (A-MO) were designed to target a 25 nucleotide sequence beginning at the start codon of the xSema3A mRNA (5′-ATG CAA TCC AGG TCA GAG AGC CCA T′) (SEQ ID NO: 8) or the xCaᵥ2.3_{α1E} mRNA (recombinant clone xCacnale, 5′-CTA TGT GAT ATG ATT ATT TAT GAC C-3′) (SEQ ID NO:7) as recommended by GeneTools. Corresponding sense sequence oligonucleotides (S-MOs) of the same regions of the xSema3A mRNA (5′-ATG-GGC-TCT-CTG-ACC-TGG-ATTGCA-T-3′) (SEQ ID NO: 9) or the xCacnale mRNA (5′-GGT CAT AAA TAA TCA TAT CAC ATA G3′) (SEQ ID NO: 10) were used as controls. Morpholino oligonucleotides (MOs, 1 mM, total 2 nl) and/or mCacnale mRNA (0.5 μg·μl⁻¹) were injected together with GFP mRNA (0.25 μg·μl¹) into one dorsal blastomere of four-cell stage embryos.

### Example 4

### Guidance Proteins and Pharmacological Drugs

Sema3A (2 U·ml⁻¹) and netrin-1 (5 ng·ml⁻¹) were prepared as described (Nishiyama et al., “Membrane Potential Shifts Caused by Diffusible Guidance Signals Direct Growth-Cone Turning,” Nat. Neurosci. 11:762-771 (2008); Togashi et al., “Cyclic GMP-Gated CNG Channels Function in Sema3A-Induced Growth Cone Repulsion,” Neuron 58:694-707 (2008); Goshima, “A Novel Action of Collapsin: Collapsin-1 Increases Antero- and Retrograde Axoplasmic Transport Independently of Growth Cone Collapse,” J. Neurobiol. 33:316-328 (1997); Hong et al., “Calcium Signalling in the Guidance of Nerve Growth by Netrin-1,” Nature 403:93-98 (2000); Nishiyama et al., “Cyclic AMP/GMP-Dependent Modulation of Ca²⁺ Channels Sets the Polarity of Nerve Growth-Cone Turning,” Nature 423:990-995 (2003); Hong et al., “A Ligand-Gated Association Between Cytoplasmic Domains of UNC5 and DCC Family Receptors Converts Netrin-Induced Growth Cone Attraction to Repulsion,” Cell 97:927-941 (1999), which are hereby incorporated by reference in their entirety). To test the conversion of the identities of individual neurites and for growth cone electrophysiology, 2,000 U/ml Sema3A (in micropipettes) was applied as a gradient. The biological activity of Sema3A (U·ml⁻¹) was evaluated using a collapse assay of chick DRG neurons (Goshima, “A Novel Action of Collapsin: Collapsin-1 Increases Antero- and Retrograde Axoplasmic Transport Independently of Growth Cone Collapse,” J. Neurobiol. 33:316-328 (1997), which is hereby incorporated by reference in its entirety). All pharmacological agents were bath-applied 30 min before experiments were performed unless otherwise indicated. Agonist and antagonists were purchased from Calbiochem, Peptide Institute, Sigma, and Tocris.

### Example 5

### Immunocytochemistry

Xenopus spinal neurons were fixed in 4% paraformaldehyde (vol/vol) for 30 min, permeabilized with 0.5% Triton X-100 (vol/vol) for 10 min, and treated with blocking solution [3% bovine serum albumin (wt/vol)] for 1 hour (Nishiyama et al., “Membrane Potential Shifts Caused by Diffusible Guidance Signals Direct Growth-Cone Turning,” Nat. Neurosci. 11:762-771 (2008); Togashi et al., “Cyclic GMP-Gated CNG Channels Function in Sema3A-Induced Growth Cone Repulsion,” Neuron 58:694-707 (2008), which are hereby incorporated by reference in their entirety). Primary antibodies were applied for 18 hours at 4° C.: II β-tubulin (1:500, 7B9, Covance), tau-1 (1:200, MAB3420, Millipore), GAP43 (1:500, Pierce Endogen) and MAP2 (1:200, AP18, Thermo Scientific). The secondary antibody, Cy3-conjugated antibody against mouse IgG (1:200, Jackson ImmunoResearch), was applied for 2 hours at 24° C. Images were captured with a confocal microscope (LSM510META, Zeiss) or an ORCA-ER CCD camera (Hamamatsu) attached to a BX51WI microscope (XLUMPlanF1, 20×0.95 NA, Olympus) and fluorescent intensities at the growth cone and proximal neurite were analyzed with the ImageJ program. To verify the specificity of tau-1 and MAP2 antibodies, immunohistochemistry was performed on 20 μm thick slice sections of Xenopus spinal cords obtained from stage 26 embryos, stage 36 tadpoles and adult animals (FIG. 4C). Animals were fixed with PLP (4% paraformaldehyde, 0.075 M lysine, 0.01 M sodium metaperiodate) solution, soaked in 30% sucrose and embedded in OCT compound (Fisher Scientific). Frozen sections were cut from embryos over a 400-μm region of the spinal cord beginning about 400 μm posterior to the back of the eyes.

### Example 6

### Electrophysiology

To measure AMPA-induced holding current shifts, an internal recording solution was used that contained the following (in mM): 140 CsMeSO4, 1 NaCl, 1 MgSO4, 1 Mg-ATP, 1 QX-314, 0.25 BAPTA and 10 HEPES (pH 7.4), and bathing solution was used that contained (in mM: 83.4 NaCl, 8.6 Na-gluconate, 2.6 KCl, 35 TEA-Cl, 2 CaCl2 and 10 HEPES; pH 7.4). Whole-cell recordings at the growth cone of cultured, CINs were made as described previously (Nishiyama et al., “Membrane Potential Shifts Caused by Diffusible Guidance Signals Direct Growth-Cone Turning,” Nat. Neurosci. 11:762-771 (2008); Togashi et al., “Cyclic GMP-Gated CNG Channels Function in Sema3A-Induced Growth Cone Repulsion,” Neuron 58:694-707 (2008); Nishiyama et al., “Cyclic AMP/GMP-Dependent Modulation of Ca²⁻ Channels Sets the Polarity of Nerve Growth-Cone Turning,” Nature 423:990-995 (2003), which are hereby incorporated by reference in their entirety). Whole-cell recordings were collected under a voltage-clamp configuration at −70 mV. Holding current shifts in response to AMPA gradients (25 mM in micropipettes) were monitored in the presence or absence of NBQX applied as a gradient (5 mM in micropipettes). Recording and bathing solutions used to measure membrane potentials (Nishiyama et al., “Membrane Potential Shifts Caused by Diffusible Guidance Signals Direct Growth-Cone Turning,” Nat. Neurosci. 11:762-771 (2008), which is hereby incorporated by reference in its entirety) and to isolate Ca²⁺ currents (Togashi et al., “Cyclic GMP-Gated CNG Channels Function in Sema3A-Induced Growth Cone Repulsion,” Neuron 58:694-707 (2008); Nishiyama et al., “Cyclic AMP/GMP-Dependent Modulation of Ca²⁺ Channels Sets the Polarity of Nerve Growth-Cone Turning,” Nature 423:990-995 (2003), which are hereby incorporated by reference in their entirety) were as described previously. Sema3A was applied as a gradient for at least five min before whole cell recordings were made. Whole-cell membrane potentials were measured under a current-clamp configuration without accessing currents. Whole-cell Ca²⁺ currents evoked by depolarization from a holding potential of −50 mV to +50 mV in 10-mV incremental steps of 100 msec each were recorded with a patch clamp amplifier (Axopatch 200B, Axon Inst.). Ca²⁺ channel activity was evaluated by measuring peak inward currents. Leak currents evoked by an inverse voltage-ramp from +120 to −120 mV were measured in the presence or absence of gradients of Sema3A and were subtracted from the total Ca²⁺ currents. Data were filtered at 2 kHz and collected at 250 Hz (membrane potentials) and 10 kHz (AMPA-induced holding potential shifts and Ca²⁺ currents).

### Example 7

### Microinjection for Imaging of Individual Neurons and to Apply Antagonists/Agonist in Embryonic Tailbud Stages

For the visualization of individual neurons, Alexa Fluor® 488 dextran, MW 10,000 (0.1 μg·μl¹) alone or together with MOs was injected into one dorsal blastomere of four-cell stage embryos followed by injection of Texas Red® dextran, MW 70,000 (1 mM) at the same injection site (as indicated by the membrane penetration mark of the previous injection) of 64-cell stage embryos. For the local administration of antagonists and agonist, the Ca²⁺ channel blockers [SNX-482 (25-45 μM), nimodipine (500 μM) and ω-conotoxin GVIA (90 μM)], ODQ (100 μM), Rp-8-pCPT-cGMPS (2.5 mM) and 8-Br-cGMP (10 mM) were injected along with FITC dextran, MW 70,000 (1 mM) into the hindbrain and at multiple sites along the spinal cord of tailbud stage 30-32 animals (total ca. 30 nl each, see FIG. 13G).

### Example 8

### Whole-Mount Immunohistochemistry

Xenopus tailbud and tadpole stage animals (control, A-MOs or S-MOs injected or overexpressing either hSema3A or mCaᵥ2.3_{α1E}) were fixed in a solution containing 4% paraformaldehyde (vol/vol) and 4% sucrose (wt/vol) for 24 hours at 4° C. Blocking solution contained 0.5% fish gelatin (wt/vol) and 0.2% Triton X-100 (vol/vol). Primary antibodies were applied at a 1:100 dilution for 24 hours at 4° C.: anti-Sema3A (AF1250, R&D Systems), anti-Npn-1 (C-19, Santa Cruz Biotechnology, Inc.), tau-1, anti-MAP2, and anti-mCaᵥ2.3_{α1E} (H-60, sc-28618, Santa Cruz Biotechnology, Inc.). Secondary antibodies were applied at a 1:100 dilution for 2 hours at 24° C.: Cy3-conjugated antibodies against mouse IgG (Jackson ImmunoResearch) for tau-1 and anti-MAP2, Cy3-conjugated antibodies against goat IgG (Jackson ImmunoResearch) for anti-Sema3A, and Cy3-conjugated antibodies against rabbit

IgG (Jackson ImmunoResearch) for anti-mCaᵥ2.3_{α1E}.

### Example 9

### In Vivo Imaging

Images of 1- or 2-μm thick z-series optical slices (150-180 or 75-90 slices, respectively) taken with an ORCA-ER CCD camera (Hamamatsu) attached to a BX51WI microscope (XLUMPlanF1, 20×0.95 NA, Olympus) were recorded using open source μManager software. Deconvolution was performed with ImageJ software using the Iterative Deconvolve 3D plugin (DAMAS3 algorithm with the Wiener filter, Optinav), and the images of each slice were used to measure volume and mean area pixel intensities with Amira (Visage Imaging, Inc), Imaris (Bitplane) or ImageJ. Flattened images of the fluorescent signals from slices that revealed the maximum intensity projections are shown in FIGS. 10B-10E; 10G-10J; 12C-12F; 13A-13E; 13H-13K; 6B-6F; 15B-15G; 15I-15N; 11C; and 16D. The average intensity projections were applied to visualize individual neurons shown in FIGS. 6I-6L. The intensities of MAP2 and tau-1 immunoreactivity (“IR”) decrease along the spinal cord in the rostral to caudal direction, coincident with the progressive developmental delay that occurs in that direction. To quantitate the IR signals, IR intensity along 300 μm of the longitudinal length of the spinal cord was measured at the position where it was ca.100 μm in diameter.

### Example 10

### Western Blotting

Spinal cords were isolated from stage 42, non-treated control Xenopus tadpoles or tadpoles injected locally with either SNX-482, ω-conotoxin GVIA, or nimodipine. Specimens were lysed with Tris-Triton lysis buffer (in mM: 100 Tris at pH 7.4, 100 NaCl, 1 EDTA, 1 EGTA, 1% Triton X-100, 10% glycerol, 0.1% SDS, 0.5% deoxycholate and protease inhibitor cocktail; AMRESCO). Proteins in these lysates were separated by a 7.5% SDS-PAGE and electro-transferred onto PVDF membranes (Millipore) as previously described (Viereck et al., “Phylogenetic Conservation of Brain Microtubule-Associated Proteins MAP2 and Tau,” Neuroscience 26:893-904 (1988), which is hereby incorporated by reference in its entirety). The membranes were divided into two sections between the 130 KD and 250 KD molecular weight markers (FERMENTAS). The sections containing the higher and lower molecular weight proteins were stained, respectively, with anti-MAP2 (1:200) or tau-1 (1:400) antibodies. The signals were detected using HRP-based ABC kit (Vector Laboratories). Used Image J to measure the densitometry of bands and analyzed the ratio of tau-1 IR to MAP2 IR of individual samples.

### Example 11

### Statistical Analysis

All the experimental data were obtained from at least three different budges of Xenopus oocytes, six different individual animals and 13 different cultured neurons. Mann-Whitney U test was used to calculate significant differences from the corresponding controls except that Wilcoxon-signed rank test was used for FIG. 7C.

### Example 12

### Sema3A Converts the Identity of Axons to Dendrites in Vitro

Unlike cultured hippocampal neurons (Craig & Banker, “Neuronal Polarity,” Annu. Rev. Neurosci. 17:267-310 (1994), which is hereby incorporated by reference in its entirety), cultured xSCINs from stage 26 embryos (Nishiyama et al., “Membrane Potential Shifts Caused by Diffusible Guidance Signals Direct Growth-Cone Turning,” Nat. Neurosci. 11:762-771 (2008); Togashi et al., “Cyclic GMP-Gated CNG Channels Function in Sema3A-Induced Growth Cone Repulsion,” Neuron 58:694-707 (2008), which are hereby incorporated by reference in their entirety) have neurites that are predominantly mono- (FIGS. 9A-9D, 9F-9I) or bi-polar (FIGS. 7A-7B), without substantial branches (FIGS. 8A-8C) or other distinguishing features of axons and dendrites. When cultured on poly-L-lysine for 16 hours, most neurons were unpolarized, immunonegative for both axonal markers tau-1 and GAP-43 and dendritic marker MAP2 (FIGS. 4A and 4B). When cultured on laminin for either three or six hours, most of these unpolarized neurites became tau-1-positive axons (Esch et al., “Local Presentation of Substrate Molecules Directs Axon Specification by Cultured Hippocampal Neurons,” J. Neurosci. 19:6417-6426 (1999), which is hereby incorporated by reference in its entirety) (FIGS. 9A and 9E). However, when these neurites were treated with Sema3A for three hours, the tau-1 immunoreactivity (IR) was markedly decreased (FIGS. 9B and 9E) while MAP2-IR was significantly increased (FIGS. 9D and 9E). Both changes were prevented by bath-applied SNP-1, the soluble Npn-1 ectodomain peptide that sequesters Sema3A (FIG. 9E) (Goshima, “A Novel Action of Collapsin: Collapsin-1 Increases Antero- and Retrograde Axoplasmic Transport Independently of Growth Cone Collapse,” J. Neurobiol. 33:316-328 (1997), which is hereby incorporated by reference in its entirety), indicating the specificity of Sema3A. Sema3A promotes the outgrowth and branching of dendrites of cortical pyramidal neurons (Fenstermaker et al., “Regulation of Dendritic Length and Branching by Semaphorin 3A,” J. Neurobiol. 58:403-412 (2004); Morita et al., “Regulation of Dendritic Branching and Spine Maturation by Semaphorin3a-Fyn Signaling,” J. Neurosci. 26:2971-2980 (2006), which are hereby incorporated by reference in their entirety) and axotomy causes conversion of preformed axons of cultured hippocampal neurons to dendrites (Bradke & Dotti, “Differentiated Neurons Retain the Capacity to Generate Axons From Dendrites,” Curr. Biol. 10:1467-1470 (2000); Gomis-Riith et al., “Plasticity of Polarization: Changing Dendrites Into Axons in Neurons Integrated in Neuronal Circuits.” Curr. Biol. 18:992-1000 (2008), which are hereby incorporated by reference in their entirety). Significant morphological changes in the length or branching of xSCIN neurites after Sema3A treatment were not observed (FIGS. 8A-8C). Netrin-1, another secreted factor that effects axon initiation in C. elegans (Adler et al., “UNC-6/Netrin Induces Neuronal Asymmetry and Defines the Site of Axon Formation,” Nat. Neurosci. 9:511-518 (2006), which is hereby incorporated by reference in its entirety), had no significant effect on the expression of tau-1- or MAP2-IR (FIG. 9E). Functional a-amino-3-hydroxy-5-methyl-4-isoxazolepropionic acid (AMPA) glutamate receptors, which are present predominantly in dendrites (Bradke & Dotti, “Differentiated Neurons Retain the Capacity to Generate Axons From Dendrites,” Curr. Biol. 10:1467-1470 (2000); Song et al., “A Selective Filter for Cytoplasmic Transport at the Axon Initial Segment,” Cell 136:1148-1160 (2009), which are hereby incorporated by reference in their entirety), were also monitored by measuring holding current shifts induced by a gradient of AMPA. Significant AMPA currents were detected in almost all growth cones treated with Sema3A, but only in less than 30% of control growth cones (FIGS. 5A-5C). Thus, Sema3A likely initiates conversion of the identity of axons to dendrites before morphological changes occur.

To determine whether conversion of the neurite identity initiates within the growth cone, a gradient of Sema3A was applied to one growth cone of bipolar neurons (FIG. 7A). The majority of those neurites that faced the source of the Sema3A became MAP2-positive dendrites but those that faced away from the source remained MAP2-negative (FIGS. 7B and 7C). Downstream signaling events induced by Sema3A in the growth cone, therefore, are likely sufficient to initiate conversion of the neurite identity.

### Example 13

### Sema3A-Induced Conversion of Neurite Identity Requires Caᵥ2.3

Guidance cues induce growth cone membrane potential shifts; attractants cause depolarization and repellents cause hyperpolarization (Nishiyama et al., “Membrane Potential Shifts Caused by Diffusible Guidance Signals Direct Growth-Cone Turning,” Nat. Neurosci. 11:762-771 (2008), which is hereby incorporated by reference in its entirety). A relatively high concentration of Sema3A induced growth cone depolarization of ca. 15 mV from the resting potential of control growth cones, but not in growth cones pre-treated with SNP-1 or that overexpressed a mutant Npn-1 [NP-1 (0111)] (Nishiyama et al., “Membrane Potential Shifts Caused by Diffusible Guidance Signals Direct Growth-Cone Turning,” Nat. Neurosci. 11:762-771 (2008); Togashi et al., “Cyclic GMP-Gated CNG Channels Function in Sema3A-Induced Growth Cone Repulsion,” Neuron 58:694-707 (2008), which are hereby incorporated by reference in their entirety) (FIGS. 3B and 3C). This 15 mV depolarization should be sufficient to cause Ca^{t}' entry through voltage-dependent Ca²⁺ channels. Functional L-, N-, R (Caᵥ2.3)- and T-type Ca²⁺ channels, but not P/Q types (Jimenez-Gonzalez et al., “Development of Ca²⁺-Channel and BK-Channel Expression in Embryos and Larvae of Xenopus laevis,” Eur. J. Neurosci. 18:2175-2187 (2003), which is hereby incorporated by reference in its entirety), have been detected in cultured embryonic Xenopus spinal neurons (Gu & Spitzer, “Low-Threshold Ca²⁺ Current and its Role in Spontaneous Elevations of Intracellular Ca²⁺ in Developing Xenopus Neurons,” J. Neurosci. 13:4936- 4948 (1993); Hong et al., “Calcium Signalling in the Guidance of Nerve Growth by Netrin-1,” Nature 403:93-98 (2000); Li et al., “Calcium Channels in Xenopus Spinal Neurons Differ in Somas and Presynaptic Terminals,” J. Neurophysiol. 86:269-279 (2001); Nishiyama et al., “Cyclic AMP/GMP-Dependent Modulation of Ca²⁻ Channels Sets the Polarity of Nerve Growth-Cone Turning,” Nature 423:990-995 (2003), which are hereby incorporated by reference in their entirety). A computed biophysical growth cone model was used (Togashi et al., “Cyclic GMP-Gated CNG Channels Function in Sema3A-Induced Growth Cone Repulsion,” Neuron 58:694-707 (2008), which is hereby incorporated by reference in its entirety) to identify those Ca²⁺ channels that would be active preferentially during Sema3A-induced membrane depolarization. This model predicted that mainly Caᵥ2.3 would conduct Ca²⁺, that T-types would undergo voltage-dependent inactivation, and that activation of L-/N-types would require further depolarization (FIG. 3D). The effect of Caᵥ2.3 on the ability of Sema3A to induce the dendrite identity was then examined. A specific Caᵥ2 3 inhibitor, SNX-482, in the bath, indeed, prevented conversion of the tau-1-positve axons to the MAP2-positive dendrites (FIGS. 9H-9J), but inhibitors of neither L-types (nimodipine) nor N-types (ω-conotoxin GVIA) prevented the conversion (FIG. 9J). Thus, in vitro, Sema3A-induced conversion of xSCIN axons to dendrites depends on Caᵥ2.3 activity.

### Example 14

### Sema3A Recruits Functional Caᵥ2.3

To determine whether Sema3A causes activation of Caᵥ2.3, Ca²⁺ currents in the growth cone were measured (Togashi et al., “Cyclic GMP-Gated CNG Channels Function in Sema3A-Induced Growth Cone Repulsion,” Neuron 58:694-707 (2008); Nishiyama et al., “Cyclic AMP/GMP-Dependent Modulation of Ca²⁺ Channels Sets the Polarity of Nerve Growth-Cone Turning,” Nature 423:990-995 (2003), which are hereby incorporated by reference in their entirety) in the presence of a gradient of Sema3A. High voltage-activated (HVA) Ca²⁺ currents were measured in the presence of bath-applied pimozide, a T-type channel blocker. N-type and Caᵥ2.3 currents were then isolated by blocking residual T- and L-type currents with nimodipine (Nishiyama et al., “Cyclic AMP/GMP-Dependent Modulation of Ca²⁺ Channels Sets the Polarity of Nerve Growth-Cone Turning,” Nature 423:990-995 (2003), which is hereby incorporated by reference in its entirety). Sema3A caused an increase in total N-type and Caᵥ2.3 currents (FIGS. 2B and 2E) without affecting the magnitude of total HVA currents (FIGS. 2A and 2E), indicating that Sema3A suppressed L-type currents. The Sema3A-induced growth cone Ca²⁺ currents was further isolated using Ni²⁺ (Caᵥ2.3 and T-type channel blocker) and nimodipine and found that N-type currents were significantly decreased (FIGS. 2C and 2E). In contrast, Sema3A caused a 4-5 fold increase in Caᵥ2.3 currents isolated in the presence of nimodipine and -conotoxin GVIA (FIGS. 2D and 2E) that were abolished by bath-applied SNX-482 (FIGS. 2D and 2E). Therefore, Sema3A caused an increase in the active pools of Caᵥ2.3 that conduct Ca²⁺ during the ca. 15 mV membrane depolarization (FIG. 3D) and suppressed L-/N-type currents. Since Sema3A did not alter the activation/inactivation time constants of the Caᵥ2.3 currents (Table 1, infra), it likely caused an increase in the number of Caᵥ2.3 in the growth cone plasma membrane rather than modulating their gating properties (FIG. 3E).

TABLE 1
Voltage-dependent activation and inactivation time constant of R-type
Ca²⁺ currents at growth cones of cultured Xenopus CINs

| | Activation t | Inactivation t |
|---|---|---|
| at + 10 mV | (msec) | (msec) |
| Control, n = 10 | 3.7 ± 0.7 | 61.5 ± 14.0 |
| Sema3A, n = 13 | 2.9 ± 0.5 | 62.2 ± 5.6 |

Data represent ± s.e.m.

### Example 15

### Expression of Caᵥ2.3 In Vivo Requires Sema3A

To examine the role of Sema3A in Caᵥ2.3 function in vivo, the effect of down-regulating endogenous Sema3A was first tested by injecting an antisense-morpholino oligonucleotide (A-MO) against Xenopus Sema3A (xSema3A) mRNA [xSema3A-A-MO; a sense-MO (xSema3A-S-MO) served as a control] into one dorsal blastomere of four-cell stage embryos along with GFP mRNA as an injection marker. Whole-mounts were then immunostained (Moon & Gomez, “Adjacent Pioneer Commissural Interneuron Growth Cones Switch From Contact Avoidance to Axon Fasciculation After Midline Crossing,” Dev. Biol. 288:474-486 (2005), which is hereby incorporated by reference in its entirety) and imaged the immuno- and GFP fluorescence (FIG. 10A) to compare the MO-injected side of the spinal cord with the uninjected side in the same embryo (FIG. 10A) and also with uninjected control embryos. Immunostaining with a polyclonal antibody against hSema3A revealed a high level of bilateral expression of xSema3A in stage 30-32 spinal cords (FIG. 3b, b′) that was reduced significantly by xSema3A-A-MO (FIG. 10C and bottom panel, labeled c′; FIG. 10F), but not by xSema3A-S-MO (FIG. 10D and bottom panel, labeled d′; FIG. 10F), attesting to the specificity of the A-MO. The reduction of xSema3A-IR was reversed by the injection of hSema3A mRNA along with xSema3AA-MO (FIG. 10E and bottom panel, labeled e′; FIG. 10F), confirming the specificity of the Sema3A antibody.

The effect of Sema3A down-regulation on the expression of Caᵥ2.3 was then examined. Immunostaining with polyclonal antibodies against hCaᵥ2.3_{α1E} revealed a pattern of expression of xCaᵥ2.3_{α1E} (xCaᵥ2.3_{α1E}) in the lateral marginal zones of the spinal cord (FIG. 10G and bottom panel, labeled g′) similar to that of xSema3A (FIG. 10B and bottom panel, labeled b′). The specificity of the anti-Caᵥ2.3_{α1E} antibodies was confirmed by their detection of over-expressed mCaᵥ2.3_{α1E} when endogenous xCaᵥ2.3_{α1E} was down-regulated by a specific A-MO (xCaᵥ2.3_{α1E}-A-MO; FIGS. 11A-11D). The endogenous expression of xCaᵥ2.3_{α1E} became prominent at stage 32-34, following the prominent expression of xSema3A at stage 30-32. Strikingly, the expression of xCaᵥ2.3_{α1E} was reduced markedly by injection of xSema3A-A-MO (FIG. 10H and bottom panel, labeled h′; FIG. 10K), but not the control xSema3A-S-MO (FIG. 10I and bottom panel, labeled i′; FIG. 10K). The effect of xSema3A-AMO was specific since over-expression of hSema3A reversed the reduction of xCaᵥ2.3_{α1E} (FIG. 10J and bottom panel, labeled j′; FIG. 10K). These results demonstrate that Sema3A is required for the expression of Caᵥ2.3 in the Xenopus spinal cord in vivo.

### Example 16

### Sema3A- Caᵥ2.3 Signaling is Required for the Dendrite Identity In Vivo

Next, it was confirmed that Sema3A signaling is required to specify the dendrite identity in vivo by analyzing MAP2-IR in whole-mount spinal cords. Dendrites of developing spinal neurons first became prominently MAP2 positive (FIGS. 12A and 12B) at the stage 42 tadpole (FIG. 12A, left). MAP2-IR showed relatively simple dendritic arbors projecting ventrally (FIG. 12B; FIG. 12C and bottom panel, labeled c′). Their morphology and the location of their soma suggest these neurons are highly likely to be either CINs or ascending interneurons (Li et al., “Axon and Dendrite Geography Predict the Specificity of Synaptic Connections in a Functioning Spinal Cord Network,” Neural Develop. 2:17 (2007), which is hereby incorporated by reference in its entirety). It was found that MAP2-IR in the Sema3A-A-MO injected (FIG. 12D and bottom panel, labeled d′; FIG. 12G), but not the xSema3A-S-MO injected (FIG. 12E and bottom panel, labeled e′; FIG. 12G) side of spinal cords was significantly reduced compared to the uninjected side of the same spinal cords, and this effect was reversed by over-expression of hSema3A (FIG. 12F and bottom panel, labeled f; FIG. 12G). Injection of A-MO against xNpn-1 (FIG. 12G) but not the control xNpn-1-S-MO (FIG. 12G) also significantly reduced the MAP2-IR in the injected side of the spinal cord. Thus, Sema3A is required to specify the dendrite identity.

Because the reduction of MAP2-IR caused by down-regulation of either xSema3A or xNpn-1 could have caused defects in cell migration (Marin et al., “Sorting of Striatal and Cortical Interneurons Regulated by Semaphorin-Neuropilin Interactions,” Science 293:872-875 (2001), which is hereby incorporated by reference in its entirety), the effects of down-regulation of xCaᵥ2.3_{α1E} on the dendrite identity was examined. Consistent with the idea that Caᵥ2.3 is a Sema3A downstream effector required to specify the dendrite identity, MAP2-IR was significantly reduced by xCaᵥ2.3_{α1E}-A-MO, to an extent similar to that caused by Sema3A-A-MO (FIG. 13A and bottom panel, labeled a′; FIG. 13F), but not by xCaᵥ2.3_{α1E}-S-MO (FIG. 13B and bottom panel, labeled b′; FIG. 13F). Injection of the mCaᵥ2.3_{α1E} mRNA together with xCaᵥ2.3_{α1E}-A-MO sufficiently reversed the reduction of MAP2-IR (FIG. 13C and bottom panel, labeled c′; FIG. 13F) resulting from injection of xCaᵥ2.3_{α1E}-A-MO alone. However, restoration of MAP2-IR by mCaᵥ2.3_{α1E} in the side of the spinal cord treated with xCaᵥ2.3_{α1E}-A-MO occurred only in the presence of endogenous xSema3A (FIG. 13C and bottom panel, labeled c′; FIG. 13F), not when xSema3A was down-regulated (FIG. 13E and bottom panel, labeled e′; FIG. 13F), suggesting other parallel signal(s) are also required to specify the dendrite identity. The effects of pharmacological blockade of Caᵥ2.3 on the acquisition of the dendrite identity were also tested by administering different doses of SNX-482 into the spinal cords of stage 30-32 tailbuds (FIG. 13G).

Administration of 45 μM SNX-482 eliminated almost all dendrites (FIG. 13K and bottom panel, labeled k′; FIG. 13L) while lower doses caused significant reductions (FIG. 131 and bottom panel, labeled i′; FIG. 13J and bottom panel, labeled j′; FIG. 13L). The local administration of nimodipine (FIG. 13H and bottom panel, labeled h′; FIG. 13L) or ω-conotoxin GVIA (FIG. 13L) had no significant effect. These results indicate that the Sema3A signal propagates through Caᵥ2.3, whose activity is required for the acquisition of the dendrite identity.

### Example 17

### Caᵥ2.3 Suppresses the Axon Identity In Vivo

Since Sema3A induced the conversion of axons of cultured xSCINs to dendrites in vitro (FIG. 9), it was then tested whether down-regulation of Caᵥ2.3 expression also affects the axon identity in vivo. Whole mounts of normal stage 42 tadpole spinal cords showed equal intensities of bilateral axon tracts along their longitudinal axis (FIG. 6A; FIG. 6B and bottom panel, labeled b′). Injection of xCaᵥ2.3_{α1E}-A-MO (FIG. 6C and bottom panel, labeled c′; FIG. 6G), but not the control xCaᵥ2.3_{α1E}-S-MO (FIG. 6D and bottom panel, labeled d′; FIG. 6G), resulted in a significant increase of tau-1-IR in the injected side of the spinal cords (bottom panels of FIGS. 6C and 6D, labeled c′ and d′, respectively), as compared with control, uninjected animals (FIG. 6C and bottom panel, labeled c′; FIG. 6G). The increased tau-1-IR was particularly noticeable close to the ventral midline (FIG. 6C and bottom panel, labeled c′) where it is normally absent (FIG. 6B and bottom panel, labeled b′), implying that the default neuronal polarization state in the absence of Caᵥ2.3 is that axons. Furthermore, local administration of SNX482 into the spinal cord caused a significant increase in tau-1-IR throughout the spinal cord (FIG. 6E and bottom panel, labeled e′; FIG. 6G). In contrast, administration of neither nimodipine (FIG. 6F and bottom panel, labeled f; FIG. 6G) nor w-conotoxin GVIA (FIG. 6G) had significant effect. Western blot analysis showed that the ratio of MAP2 to tau-1 in total protein extracted from the SNX-482-treated spinal cords was significantly lower than that from untreated, control, spinal cords (FIGS. 14A-14C), corroborating the immunohistochemical observations of spinal cords shown above. Thus, Caᵥ2.3 activity is required not only for acquisition of the dendrite identity but also for suppression of the axon identity.

To exclude off-target effects of A-MO treatment, the neuronal integrity of xCaᵥ2.3_{α1E}-A-MO-treated animals was then examined. Texas Red conjugated with 70K-dextran was microinjected into one dorsal blastomere of 64-cell stage embryos into which xCaᵥ2.3_{α1E}-AMO had previously been injected at the four-cell stage (FIG. 6H). Individual spinal neurons in the MO-injected side of stage 42 tadpole spinal cords were then visualized by their Texas Red. Isolated, putative CINs whose soma were located ca. two thirds of the distance from the ventral midline and whose single axon and dendrites projected ventrally were imaged (FIGS. 6I and 6K, control). Individual putative CINs in the side of spinal cords treated with xCaᵥ2.3-A-MO, indeed, showed multiple axon-like neurites that emanated from the proximal segment and many that projected longitudinally in the ipsilateral axon tract (FIGS. 6J and 6 L), but such axon-like neurites were only rarely observed in control animals (FIGS. 6I and 6K). The ipsilateral projection (FIGS. 6J and 6L) of these putative CIN axons may be a result of their reduced sensitivity to netrin-1 (Stein & Tessier-Lavigne, “Hierarchical Organization of Guidance Receptors: Silencing of Netrin Attraction by Slit Through a Robo/DCC Receptor Complex,” Science 91:1928-1938 (2001), which is hereby incorporated by reference in its entirety). These results clearly demonstrate that the activity of Caᵥ2.3 suppresses the axon identity.

### Example 18

### Cyclic GMP-Dependent Expression of Caᵥ2.3 and PKG-Dependent Acquisition of the Dendrite Identity

It was previously shown that Sema3A triggers the local production of cGMP by sGC (Nishiyama et al., “Membrane Potential Shifts Caused by Diffusible Guidance Signals Direct Growth-Cone Turning,” Nat. Neurosci. 11:762-771 (2008), which is hereby incorporated by reference in its entirety). A recent study showed that cGMP-PKG signaling induces initiation of the dendrite identity in undifferentiated neurites of cultured hippocampal neurons (Shelly et al., “Local and Long-Range Reciprocal Regulation of cAMP and cGMP in Axon/Dendrite Formation,” Science 327:547-552 (2010), which is hereby incorporated by reference in its entirety). Interestingly, bath-application of 1H-[1,2,4]oxadiazolo[4,3-a]quinoxalin-1-one (ODQ), an inhibitor of sGC, prevented the induction of Caᵥ2.3 currents by a gradient of Sema3A in xSCIN growth cones (FIG. 15A). Moreover, inhibition of sGC by local administration of ODQ abolished not only Caᵥ2.3_{α1E} expression (FIG. 15B and bottom panel, labeled as b′; FIG. 15H), but also dendrite formation (FIG. 15E and bottom panel, labeled as e′; FIG. 15H), suggesting that cGMP signaling functions as an upstream regulator of the expression of Caᵥ2.3 as well as acquisition of the dendrite identity. Surprisingly, a gradient of Rp-8-pCPT-cGMPS, a PKG-inhibiting cGMP analogue (Nishiyama et al., “Membrane Potential Shifts Caused by Diffusible Guidance Signals Direct Growth-Cone Turning,” Nat. Neurosci. 11:762-771 (2008), which is hereby incorporated by reference in its entirety) induced Caᵥ2.3 currents (FIG. 15A) and pre-empted their induction by Sema3A (FIG. 15A). Moreover, Rp-8- pCPT-cGMPS-induced Caᵥ2.3 currents were not affected by ODQ, concordant with the notion that cGMP is required to target functional Caᵥ2.3 to the plasma membrane. Importantly, these Caᵥ2.3 currents were eliminated by pretreatment with cycloheximide (CHX), a protein synthesis inhibitor (FIG. 15A), suggesting that cGMP, not PKG, mediates both the Sema3A-induced synthesis of Caᵥ2.3 proteins and their targeting to the plasma membrane in vitro. Rp-8-pCPT-cGMPS also caused a significant increase in the expression of xCaᵥ2.3_{α1E} (FIG. 15C and bottom panel, labeled as c′; FIG. 15H), which extended beyond the marginal zones of the spinal cord. It is noteworthy that pre-treatment with CHX eliminated the Sema3A-induced expression of Caᵥ2.3 both in vitro and in vivo (FIGS. 16A-16E), supporting the idea that cGMP regulates the expression of functional Caᵥ2.3 at the translational level. In contrast, Rp-8-pCPT-cGMPS drastically reduced MAP2-IR (FIG. 15F and bottom panel, labeled as f′; FIG. 15H) to an extent similar to that caused by treatment with either ODQ (FIG. 15E and bottom panel, labeled as e′; FIG. 15H) or SNX-482 (FIGS. 13I-13L), demonstrating that PKG activity is required for the acquisition of the dendrite identity. Consistent with these observations, local administration of 8-BrcGMP, a PKG-activating cGMP analogue, enhanced both the expression of xCaᵥ2.3_{α1E} (FIG. 15D and bottom panel, labeled as d′; FIG. 15H) and dendrite formation (FIG. 15G and bottom panel, labeled as g′; FIG. 15H).

Finally, the expression of either xSema3A or xCaᵥ2.3_{α1E} was down-regulated with specific A-MOs in one side of spinal cords and tested the effects of varying the levels of cGMP on the expression of xCaᵥ2.3_{α1E} or acquisition of the dendrite identity. Despite the absence of Sema3A, local administration of either Rp-8-pCPT-cGMPS (FIG. 15J and bottom panel, labeled as j′; FIG. 15O) or 8-Br-cGMP (FIG. 15K and bottom panel, labeled as k′; FIG. 15O) induced the expression of xCaᵥ2 while local administration of ODQ caused their significant reduction (FIG. 15I and bottom panel, labeled as i′; FIG. 15O), supporting the idea that cGMP induces the expression of xCaᵥ2.3_{α1E}. In contrast, ODQ (FIG. 15L and bottom panel, labeled as 1′; FIG. 150) or Rp-8-pCPT-cGMPS (FIG. 15M and bottom panel, labeled as m′; FIG. 15O) prevented the appearance of dendrites even in the presence of the xCaᵥ2.3. However, local administration of 8-Br-cGMP failed to rescue dendrites in the absence of xCaᵥ2.3 (FIG. 15N and bottom panel, labeled as n′; FIG. 15O), indicating that Caᵥ2.3 is required for the acquisition of the dendrite identity. Taken together, these results reveal that Sema3A induced cGMP-PKG signaling regulates the cGMP-dependent expression of functional xCaᵥ2.3 that together with PKG as a co-effector specify the dendrite identity while suppressing axon identity.

### Example 19

### Semaphorin 3A induces Caᵥ2.3 Channel-Dependent Conversion of Axons to Dendrites

Extracellular factors signal to regulate the initiation and maintenance of the axon identity (Adler et al., “UNC-6/Netrin Induces Neuronal Asymmetry and Defines the Site of Axon Formation,” Nat. Neurosci. 9:511-518 (2006); Yi et al., “TGF-β Signaling Specifies Axons During Brain Development,” Cell 142:144-157 (2010); Shelly et al., “LKB1/STRAD Promotes Axon Initiation During Neuronal Polarization,” Cell 129:565-577 (2007), which are hereby incorporated by reference in their entirety) and dendrite growth and branching (Fenstermaker et al., “Regulation of Dendritic Length and Branching by Semaphorin 3A,” J. Neurobiol. 58:403-412 (2004); Morita et al., “Regulation of Dendritic Branching and Spine Maturation by Semaphorin3a-Fyn Signaling,” J. Neurosci. 26:2971-2980 (2006); Tran et al., “Secreted Semaphorins Control Spine Distribution and Morphogenesis in the Postnatal CNS,” Nature 462:1065-1069 (2009), which are hereby incorporated by reference in their entirety). However, the signaling events that regulate the acquisition of an identity as a dendrite in response to extracellular factors are unknown. These results demonstrate in vitro and in vivo that a secreted guidance factor, Sema3A, induces the dendrite identity and concomitantly suppresses the axon identity by inducing the expression of functional Caᵥ2.3. It is further demonstrated that Sema3A-triggered cGMP production and PKG activity are required, respectively, for the expression of Caᵥ2.3 and acquisition of the dendrite identity.

It was found that the relatively high Sema3A concentration required to convert the axons of cultured xSCINs to dendrites caused growth cone depolarization and the recruitment of functional Caᵥ2.3 to the plasma membrane. It was also found that increased expression of Sema3A occurs in close spatiotemporal proximity to the expression of Caᵥ2.3 in the spinal cords of tailbud stage animals, when putative dendrites emanate from pre-existing axons. The results described herein indicate that a high Sema3A concentration is required to induce Caᵥ2.3-mediated Ca²⁺ signaling in growth cones, which, in turn, causes the up-regulation of MAP2 required to specify the dendrite identity while simultaneously down-regulating the unphosphorylated tau required to specify the axon identity (see FIG. 17). Local protein synthesis and trafficking can occur in cultured growth cones of Xenopus retinal ganglion cells (xRGCs) within 5 minutes of their exposure to Sema3A (Piper et al., “Endocytosis-Dependent Desensitization and Protein Synthesis-Dependent Resensitization in Retinal Growth Cone Adaptation,” Nat. Neurosci. 8:179-186 (2005), which is hereby incorporated by reference in its entirety). Thus, without being bound by theory, Sema3A signaling may induce cGMP-dependent de novo synthesis of Caᵥ2.3 and their targeting to the plasma membrane. Sema3A has been demonstrated to activate the ERK (extracellular-signal-regulated-kinase)-TOR (target-of-rapamycin) signaling pathway during repulsion/collapse of cultured xRGC growth cones (Campbell & Holt, “Chemotropic Responses of Retinal Growth Cones Mediated by Rapid Local Protein Synthesis and Degradation,” Neuron 32:1013-1026 (2001); Campbell & Holt, “Apoptotic Pathway and MAPKs Differentially Regulate Chemotropic Responses of Retinal Growth Cones,” Neuron 37:939-952 (2003), which are hereby incorporated by reference in their entirety). CNrasGEF, a cyclic nucleotide-activated guanine exchanging factor that activates GTPase Ras, could couple Sema3A-induced production of cGMP to the ERK-TOR signaling pathway (Pham et al., “The Guanine Nucleotide Exchange Factor CNrasGEF Activates Ras in Response to cAMP and cGMP,” Curr. Biol. 10:555-558 (2000), which is hereby incorporated by reference in its entirety) to induce de novo synthesis of Caᵥ2.3. A similar mechanism could account for the cGMP mediated targeting of Caᵥ2.3 to the plasma membrane, since Ras/Rap1 are known to induce vesicle transport that traffics the ionotropic glutamate receptor (Stornetta & Zhu, “Ras and Rap Signaling in Synaptic Plasticity and Mental Disorders,” Neuroscientist 17:54-78 (2011), which is hereby incorporated by reference in its entirety), and Sema3A enhances axonal vesicle transport (Goshima, “A Novel Action of Collapsin: Collapsin-1 Increases Antero- and Retrograde Axoplasmic Transport Independently of Growth Cone Collapse,” J. Neurobiol. 33:316-328 (1997), which is hereby incorporated by reference in its entirety).

The identity of neurites as either axons or dendrites depends on the relative signaling of cAMP-PKA and cGMP-PKG; greater cGMP-PKG signaling causes undifferentiated neurites of hippocampal neurons to become dendrites (Shelly et al., “Local and Long-Range Reciprocal Regulation of cAMP and cGMP in Axon/Dendrite Formation,” Science 327:547-552 (2010), which is hereby incorporated by reference in its entirety). PKG antagonizes acquisition of the axon identity induced by PKA-dependent phosphorylation of LKB1 (serine/threonine kinase 11) (Shelly et al., “LKB1/STRAD Promotes Axon Initiation During Neuronal Polarization,” Cell 129:565-577 (2007); Barnes et al., “LKB1 and SAD Kinases Define a Pathway Required for the Polarization of Cortical Neurons,” Cell 129:549-563 (2007), which are hereby incorporated by reference in their entirety) and glycogen synthase kinase-3β (GSK3β) (Jiang et al., “Both the Establishment and the Maintenance of Neuronal Polarity Require Active Mechanisms: Critical Roles of GSK-3J3 and Its Upstream Regulators,” Cell 120:123-135 (2005), which is hereby incorporated by reference in its entirety). Sema3A-induced PKG activity (Polleux et al., “Semaphorin 3A is a Chemoattractant for Cortical Apical Dendrites,” Nature 404:567-573 (2000), which is hereby incorporated by reference in its entirety) may gate Ca²⁺ entry through Caᵥ2.3 by inducing membrane depolarization (Nishiyama et al., “Membrane Potential Shifts Caused by Diffusible Guidance Signals Direct Growth-Cone Turning,” Nat. Neurosci. 11:762-771 (2008), which is hereby incorporated by reference in its entirety) and simultaneously opposing the PKA activity required for the axon identity.

The details of the mechanism that underlies the activation of Caᵥ2.3 required for the acquisition of the dendrite identity is unknown, but may require an unidentified factor(s) (Factor “X”, FIG. 17). This hypothesized factor is unlikely any of the known signaling molecules that promote the axon identity because disruption of the expression of these molecules does not cause the conversion of axons to dendrites. Sema3A causes the phosphorylation of collapsing-response-mediator-protein-2 (CRMP2) through the action of cyclin-dependent-kinase-5 and GSK3β (Uchida et al., “Semaphorin3A Signalling is Mediated Via Sequential Cdk5 and GSK3β Phosphorylation of CRMP2: Implication of Common Phosphorylating Mechanism Underlying Axon Guidance and Alzheimer's Disease,” Genes Cells 10:165-179 (2005), which is hereby incorporated by reference in its entirety) that is known to suppress the axon identity (Yoshimura et al., “GSK-3J3 Regulates Phosphorylation of CRMP-2 and Neuronal Polarity,” Cell 120:137-149 (2005), which is hereby incorporated by reference in its entirety). Whether Caᵥ2.3 functions by modulating the phosphorylation states of either tau-1 (Liu et al., “Involvement of Aberrant Glycosylation in Phosphorylation of Tau by cdk5 and GSK-3β,” FEBS Lett. 530:209-214 (2002), which is hereby incorporated by reference in its entirety) or CRMP2 (Yoshimura et al., “GSK-3J3 Regulates Phosphorylation of CRMP-2 and Neuronal Polarity,” Cell 120:137-149 (2005), which is hereby incorporated by reference in its entirety) requires further study.

Although the localization of Caᵥ2.3 predominantly to dendrites (Magee & Johnston, “Characterization of Single Voltage-Gated Na⁺ and Ca²⁺ Channels in Apical Dendrites of rat CA1 Pyramidal Neurons,” J. Physiol. 487:67-90 (1995), which is hereby incorporated by reference in its entirety) and spine heads (Sabatini & Svoboda, “Analysis of Calcium Channels in Single Spines Using Optical Fluctuation Analysis,” Nature 408:589-593 (2000), which is hereby incorporated by reference in its entirety) has been demonstrated, their function in vivo has not been elucidated previously. This study demonstrates the importance of the spatiotemporal and dynamic expression patterns of both guidance factors and ion channels in establishing neurite identity during nervous system development. Caᵥ2.3-induced acquisition of the dendrite identity and the simultaneous suppression of the axon identity may also contribute to the Sema3Ainduced failure of axons to regenerate after nerve injury (Kaneko et al., “A Selective Sema3A Inhibitor Enhances Regenerative Responses and Functional Recovery of the Injured Spinal Cord,” Nat. Med. 12:1380-1389 (2006), which is hereby incorporated by reference in its entirety).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of controlling axon or dendrite development in a neuronal cell population comprising:
providing a neuronal cell population and
contacting the neuronal cell population with a modulator of R-type Ca²⁺ channel expression or activity in the neuronal cell population to induce either axon or dendrite development.

2. The method according to claim 1, wherein the modulator is an inhibitor of R-type Ca²⁺ channel expression or activity which induces axon development in the neuronal cell population.

3. The method according to claim 2, wherein the modulator is an inhibitor of R-type Ca²⁺ channel expression and directly interacts with a nucleic acid molecule encoding the R-type Ca²⁺ channel.

4. The method according to claim 2, wherein the modulator is an inhibitor of R-type Ca²⁺ channel activity and directly interacts with the R-type Ca²⁺ channel.

5. The method according to claim 2, wherein the inhibitor is selected from the group consisting of a nucleic acid molecule, an inhibitory peptide, an antibody, and a small molecule.

6. The method according to claim 2, wherein the inhibitor is SNX-482.

7. The method according to claim 1, wherein the modulator is an agent that induces R-type Ca²⁻ channel expression or activity which induces dendrite development in the neuronal cell population.

8. The method according to claim 7, wherein the agent comprises semaphorin 3A (“Sema3A”), cGMP, or both Sema3A and cGMP.

9. The method according to claim 1, wherein said contacting is carried out in vivo.

10. The method according to claim 1, wherein said contacting is carried out in vitro.

11. The method according to claim 1, wherein the R-type Ca²⁺ channel is a Caᵥ2.3 channel.

12. The method according to claim 1, wherein said providing comprises:
providing a population of pluripotent stem cells and
inducing production of neurons from the population of pluripotent stem cells.

13. The method according to claim 1, wherein said contacting comprises:
treating said cell population with a modulator of R-type Ca²⁺ channel expression or activity that induces axon development and contacting the treated neuronal cell population with a modulator of R-type Ca²⁺ channel expression or activity that induces dendrite development.

14. The method according to claim 13, wherein the modulator of R-type Ca²⁺ channel expression or activity that induces dendrite development comprises Sema3A, cGMP, or both Sema3A and cGMP.

15. The method according to claim 13, wherein said contacting the treated neuronal cell population is carried out in vivo.

16. The method according to claim 13, wherein said contacting is carried out in vitro.

17. A method of treating neuronal injury in a subject, said method comprising:
selecting a subject with neuronal injury mediated by R-type Ca²⁺ channel expression or activity and
administering to the selected subject an inhibitor of R-type Ca²⁺ channel expression or activity to induce neuronal axon development under conditions effective to treat the neuronal injury in the subject.

18. The method according to claim 17, wherein the neuronal injury is associated with amyotrophic lateral sclerosis (ALS), trigeminal neuralgia, glossopharyngeal neuralgia, Bell's Palsy, myasthenia gravis, muscular dystrophy, progressive muscular atrophy, primary lateral sclerosis (PLS), pseudobulbar palsy, progressive bulbar palsy, spinal muscular atrophy, inherited muscular atrophy, invertebrate disk syndromes, cervical spondylosis, plexus disorders, thoracic outlet destruction syndromes, peripheral neuropathies, prophyria, Alzheimer's disease, Huntington's disease, Parkinson's disease, Parkinson-plus syndrome, multiple system atrophy, progressive supranuclear palsy, corticobasal degeneration, dementia with Lewy bodies, frontotemporal dementia, demyelinating diseases, Guillain-Barré syndrome, multiple sclerosis, Charcot-Marie-Tooth disease, prion disease, Creutzfeldt-Jakob disease, Gerstmann-Sträussler-Scheinker syndrome (GSS), fatal familial insomnia (FFI), bovine spongiform encephalopathy, Pick's disease, epilepsy, AIDS dementia complex, peripheral neuropathy or neuralgia, glaucoma, lattice dystrophy, retinitis pigmentosa, age-related macular degeneration (AMD), photoreceptor degeneration associated with wet or dry AMD, other retinal degeneration, optic nerve drusen, optic neuropathy, optic neuritis, traumatic nerve injury, schizophrenia, Tuberous Sclerosis complex, Autism Spectrum Disorder, or combinations thereof.

19. The method according to claim 17, wherein the inhibitor inhibits R-type Ca²⁺ channel expression by directly interacting with a nucleic acid molecule encoding the R-type Ca²⁺ channel.

20. The method according to claim 17, wherein the inhibitor is an inhibitor of R-type Ca²⁺ channel activity that directly interacts with the R-type Ca²⁺ channel.

21. The method according to claim 17, wherein the inhibitor is an inhibitor of Caᵥ2.3 channel activity or expression.

22. The method according to claim 17, wherein the inhibitor is selected from the group consisting of a nucleic acid molecule, an inhibitory peptide, an antibody, and a small molecule.

23. The method according to claim 17, wherein the inhibitor is SNX-482.

24. The method according to claim 17, wherein the inhibitor is an inhibitor of Sema3A.

25. The method according to claim 17, wherein the selected subject is a mammal.

26. The method according to claim 17, wherein the selected subject is a human.

27. The method according to claim 17, wherein said administering is carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes.

28. The method according to claim 17 further comprising:
administering to the selected subject a central nervous system (“CNS”) therapeutic in conjunction with said administering the inhibitor of R-type Ca²⁺ channel expression or activity.

29. The method according to claim 28, wherein the CNS therapeutic comprises a population of stem cells.

30. The method according to claim 28, wherein the CNS therapeutic comprises an inhibitor of Sema3A.

31. The method according to claim 17, wherein said administering the inhibitor of R-type Ca²⁺ channel expression or activity comprises:
contacting one or more neuronal cells at the neuronal injury site with an inhibitor of R-type Ca²⁺ channel expression or activity, thereby stimulating axon development in the one or more neuronal cells at the neuronal injury site.

32. A method of screening for agents that modulate R-type Ca²⁺ channel expression or activity, said method comprising:
providing a neuronal cell population;
providing one or more candidate agents;
contacting the neural cell population with the one or more candidate agents;
detecting, following said contacting, either axonal outgrowth or dendritic outgrowth by neuronal cells in the neuronal cell population; and
identifying the one or more candidate agents as agents that modulate R-type Ca²⁺ channel expression or activity, wherein, based on said detecting, increased axonal outgrowth or dendritic outgrowth is detected compared to when the neuronal cell population is not contacted with the one or more candidate agents.

33. The method according to claim 32, wherein when an increase in axon outgrowth is detected, the candidate agent is identified as an inhibitor of R-type Ca²⁻ channel expression or activity.

34. The method according to claim 32, wherein when an increase in dendrite outgrowth is detected, the candidate agent is identified as an agent that induces of R-type Ca²⁺ channel expression or activity.

35. The method according to claim 32, wherein said detecting comprises:
contacting the neuronal cell population with a reagent suitable to detect one or more markers of axonal and/or dendritic outgrowth.

36. The method according to claim 35, wherein the reagent is an antibody

37. The method according to claim 35, wherein the reagent is suitable to detect one or more markers of axonal outgrowth, wherein the one or more markers comprise tau-1, GAP-43, or both tau-1 and GAP-43.

38. The method according to claim 35, wherein the reagent is suitable to detect one or more markers of dendritic outgrowth, wherein the one or more markers comprise MAP2.

39. The method according to claim 32, wherein said neuronal cell population is a mammalian neuronal cell population.

40. The method according to claim 32, wherein said neuronal cell population is a Xenopus neuronal cell population.

41. The method according to claim 32, wherein the method is carried out in vivo.

42. The method according to claim 32, wherein the method is carried out in vitro.
